(19) United States
(12) Patent Application Publication
(10) Pub. No.: US 2007/0025914 A1
(43) Pub. Date: Feb. 1, 2007

(54) Bronchioalveolar stem cells and uses thereof

(76) Inventors: Tyler Jacks, West Newton, MA (US); Carla F. Kim, Boston, MA (US)

Correspondence Address: CLARK & ELBING LLP, 101 FEDERAL STREET, BOSTON, MA, 02110, US

(21) Appl. No.: 11/453,787

(22) Filed: Jun. 15, 2006

Related U.S. Application Data
(60) Provisional application No. 60/692,004, filed on Jun. 16, 2005.

(51) Int. Cl. A61K 51/00; G01N 33/567; C12N 5/08

(52) U.S. Cl. 424/1.49; 435/7.2; 435/366

(57) ABSTRACT

The present invention features stems cells from lungs, methods of isolating such stem cells, methods of diagnosing lung diseases, and method of treating lung diseases, including cancer, chronic obstructive pulmonary disease, cystic fibrosis, and emphysema.

### CROSS-REFERENCE TO RELATED APPLICATIONS

This application claims the benefit of U.S. provisional application No. 60/692,004, filed Jun. 16, 2005, which is hereby incorporated by reference.

### STATEMENT AS TO FEDERALLY SPONSORED RESEARCH

The present research was supported by a grant from the National Institutes of Health and National Cancer Institute (Number 6896861). The U.S. Government may therefore have certain rights to this invention.

### BACKGROUND OF THE INVENTION

For lung cancer, it is imperative to understand the earliest cellular and molecular changes to improve cancer detection and therapy. Lung cancer is the leading cause of death from cancer worldwide. The five-year survival rate for lung cancer patients with distant metastasis is only 2%. However, the survival rate for patients with early-stage, localized lung cancer is 50%. Therefore, studies to facilitate early detection are clearly predictive to make a great difference for patients, and identification of targets for cancer therapy is essential.

Previously, no screening method for early stage lung cancer existed. A large trial is underway to assess the use of CT scans of at-risk patients. However, a specific molecular detection method could better distinguish between tumor and normal cells. Current types of lung cancer therapy are lethal to normal cells as well as tumor cells. Furthermore, chemotherapeutics may fail to target tumor stem cells, thereby providing an initial response followed by reoccurrence of the tumor.

Thus, there is a need to improve detection and therapy for lung disease, including lung cancer.

### SUMMARY OF THE INVENTION

The present invention relies on our discovery of bronchiolalveolar stem cells (BASCs) and their involvement in cellular proliferation and tissue regeneration. Based on these discoveries, the present invention features methods for isolating BASCs, populations of BASCs, antibodies specific for BASCs, methods for identifying compounds that alter BASC proliferation, methods for identification of changes in BASC proliferation in a subject, and methods for treating a subject either by administration of a compound that alters BASC proliferation or by administration of a BASC.

Accordingly, in a first aspect, the invention features a method of isolating BASCs, the method including (a) isolating cells from a lung of an organism (e.g., mouse or human); (b) selecting (e.g., using FACS) from the cells a population which lacks markers for both hematopoietic (e.g., CD45, Ter119) and endothelial cells (e.g., Pecam); and (c) selecting, from the population from (b), a second population of cells containing a stem cell marker (e.g., Sca-1, CD34, SP-C, or CCA), thereby isolating BASCs. The method may further include removal of red blood cells by lysis. In another embodiment, the method may further include (d) selecting from the second population a third population using an additional marker (e.g., CD34, Sca-1). In a further embodiment, the method may include (d) growing the cells selected in (c) in a feeder cell culture (e.g., and allowing the cells to differentiate).

In another aspect, the invention features a multipotent stem cell isolated from lung, where the stem cell expresses at least one (e.g., two, three, or four) of Sca-1, SP-C, CCA, and CD34. The stem cell may include a genetic modification.

In another aspect, the invention features an antibody that specifically binds a stem cell of the previous aspect.

In another aspect, the invention features a method of identifying an increase or decrease in the number of BASCs in a lung of a subject (e.g., a human), including (a) administering (e.g., intravenously, intratracheally) to the subject a compound, for example, an antibody or fragment thereof (e.g., a CD34 antibody, or a monoclonal or polyclonal antibody specific to BASC cells) that specifically binds a BASC; and (b) detecting the compound in the lung of the subject, where an increase or decrease in the amount of the compound in the lungs of the subject as compared to an amount in a control subject indicates an increase or decrease in the number of BASCs in the lung of the subject. The detecting may be performed using an imaging technique (e.g., planar scan, PET, SPECT, MRI, and CT).

In yet another aspect, the invention features a method of identifying a candidate compound (e.g., a compound from a chemical library) for treating a subject with a disease, e.g., lung cancer or any lung disease described herein, associated with a change in BASC (e.g., a mammalian or human cell) proliferation, the method including (a) contacting a BASC with a compound; and (b) measuring proliferation and/or differentiation of the BASC, where an increased or decreased level of BASC proliferation and/or differentiation in the presence of the compound relative to the level in the absence of the compound identifies the compound as a candidate compound for treating a subject with a change in BASC proliferation. The increased level of BASC proliferation may be the result of an increased rate of cellular division, a decrease in apoptotic death, or a decrease in necrotic death. The decreased level of BASC proliferation is the result of a decreased rate of cellular division, toxicity to rapidly dividing cells, an increase in apoptotic death, or an increase in necrotic death. The method may be performed in vivo (e.g., in a mouse) or in vitro.

The invention further features a method of treating lung disease (e.g., lung cancer) in a subject, the method including administration (e.g., intratracheal or intravenous) to the subject (e.g., a human) a compound that increases or decreases proliferation and/or differentiation of BASCs in an amount sufficient to treat said lung disease. The lung cancer may be a small cell lung cancer or a non-small cell lung cancer (e.g., adenocarcinoma, a squamous cell carcinoma, a large cell carcinoma, or mixture), or a bronchial carcinoid. The method may be performed in conjunction with administering to the patient an additional treatment (e.g., surgery, radiation therapy, chemotherapy, immunotherapy, anti-angiogenesis therapy, or gene therapy) for lung cancer, where the method and the additional treatment are administered within six months of each other.

The invention also features a method of treating a subject (e.g., a human) with lung disease (e.g., chronic, obstructive pulmonary disease, cystic fibrosis, or emphysema), the method including administration (e.g., intratracheal) of BASCs (e.g., isolated BASCs or human BASCs) to the subject (e.g., in an amount sifficient to treat said disease).

By “isolated,” “substantially purified,” or “substantially isolated” is meant that the desired cells (e.g., BASCs) are enriched by at least 30%, more preferably by at least 50%, even more preferably by at least 75%, and most preferably by at least 90% or even 95%.

By “biological sample” or “sample” is meant a sample obtained from an organism or from components (e.g., cells) of an organism. The sample may be of any biological tissue or fluid. Frequently the sample will be a “clinical sample” which is a sample derived from a subject. Such samples include, but are not limited to, sputum, blood, blood cells (e.g., white cells), tissue or fine needle biopsy samples, urine, peritoneal fluid, and pleural fluid, or cells. Biological samples may also include sections of tissues such as frozen sections taken for histological purposes.

By “subject” is meant either a human or non-human animal.

“Treating” a disease or condition in a subject or “treating” a subject having a disease or condition refers to subjecting the individual to a pharmaceutical treatment, e.g., the administration of a drug, such that at least one symptom of the disease or condition is decreased, stabilized, or prevented.

By “specifically binds” or “specific binding” is meant a compound or antibody which recognizes and binds a polypeptide or cell of the invention but which does not substantially recognize and bind other molecules in a sample, for example, a biological sample, which naturally includes a polypeptide of the invention.

By “decrease” is meant a reduction of at least 5%, 10%, 25%, 50%, 75%, 80%, or even 90% of the level as compared to the number, activity, or expression under control conditions.

By “increase” is meant a positive change of at least 5%, 10%, 25%, 50%, 75%, 80%, 100%, 200%, or even 500% or more over the level as compared to the number, activity, or expression under control conditions.

By a “compound,” “candidate compound,” or “factor” is meant a chemical, be it naturally-occurring or artificially-derived. Compounds may include, for example, peptides, polypeptides, synthetic organic molecules, naturally-occurring organic molecules, nucleic acid molecules, and components or combinations thereof.

By “purified antibody” is meant antibody which is at least 60%, by weight, free from proteins and naturally-occurring organic molecules with which it is naturally associated. Preferably, the preparation is at least 75%, more preferably 90%, and most preferably at least 99%, by weight, antibody. A purified antibody of the invention may be obtained, for example, by affinity chromatography using a recombinantly-produced polypeptide of the invention and standard techniques.

Other features and advantages of the invention will be apparent from the following Detailed Description, the drawings, and the claims.

### BRIEF DESCRIPTION OF THE DRAWINGS

FIGS. 1A-1D are images showing identification of BASCs in normal lung. BASCs (arrows) are located at the bronchioalveolar duct junction (BADJ, brackets), the branch point between a terminal bronchiole (TB) lined with Clara cells positive for CCA and the alveolar space (AS) lined with alveolar epithelium including AT2 cells positive for SP-C. IF for DAPI (blue), CCA (red), and SP-C (green). FIG. 1A is a 400× image, merge. FIGS. 1B-1D are a zoom of 400× image to highlight the BADJ region. Merge (1B), green channel only (1C), and red channel only (ID). Scale bars, 10μm.

FIG. 2A-2G are images and graphs showing BASCs are damage-resistant and proliferate during epithelial repair. FIGS. 2A and 2B show IF as in FIG. 1, showing a TB from a naphthalene (2A) or mock (2B) treated mouse 36 hr after treatment. Arrows indicate BASCs; asterisks indicate Clara cells sloughing off into the airway. Inset, enlarged BADJ region. All scale bars, 10 μm. FIG. 2C shows the percentage of TBs containing 0, 1, and 2 or more BASCs at various time points after naphthalene is shown (mean±SD). FIG. 2D shows four-color IF was performed to analyze proliferation after naphthalene treatment. DAPI staining (not shown) was used to identify the outline of TBs (dashed line). A TB 52 hr after naphthalene is shown with a single BrdU^{pos} (blue), SP-C^{pos} (green), CCA^{pos} (red) BASC at the BADJ (arrow). Arrowheads point to a BrdU^{neg}, Clara cell more proximal in the TB (left) and a BrdU^{neg} BASC at the other BADJ (right). Asterisk, green autofluorescence. FIG. 2E shows staining as in FIG. 2D of a TB 72 hr after naphthalene, which shows BrdU^{neg} BASCs at the BADJ (arrowheads) situated next to BrdU^{pos} CCApos SPC^{neg} cells (arrows). Asterisks indicate cells sloughing off into the airway. FIG. 2F shows the percentage of BrdU^{pos} cells at the BADJ contributed by SP-C^{pos} CCA^{pos} cells (BASCs) or SP-C^{neg} CCA^{pos} cells (Clara cells) at various time points after naphthalene treatment is shown. Data are from at least 100 BrdU-positive cells for each time point. FIG. 2G shows the percentage of TBs containing 0, 1, and 2 or more BASCs (mean±SD) at various time points after bleomycin is shown.

FIGS. 3A and 3B show FACS methodology to isolate BASCs. FIG. 3A shows a typical scatter plot of the distinct cellular populations in lung identified by staining for Sca-1, CD45, and Pecam. The percentage of total lung represented by each population is indicated. Histograms show the percentage of cells positive for CD34 in a negative control (no CD34 antibody) (top) and in the Sca-1^{pos} CD45^{neg} Pecam^{neg} population (bottom). FIG. 3B shows IF (as in other figures) of the indicated populations for CCA and SP-C after sorting and cytospin. The percentage of cells exhibiting the representative staining pattern is 100% unless otherwise indicated. autofl^{hi}, cells with high green autofluorescence without Sca-1 staining. Scale bar, 10 μm.

FIGS. 4A-4T show that BASCs self-renew and are multipotent. FIG. 4A shows phase-contrast images of colonies resulting from (left to right) feeder-cultured BASCs, secondary BASC culture, a single Sca-1^{pos} CD45^{neg} Pecam^{neg} CD34^{pos} cell plated on day 0, and its resulting colony after 1 week. FIG. 4B shows limiting dilution analysis comparing AT2 cells, total unsorted lung, and BASCs. The percentage of wells without colony formation for the corresponding number of plated cells is shown with the coefficient and formula from regression analysis. Data from three independent experiments (mean±SD) were used. FIG. 4C shows limiting dilution analysis of serially passaged cells. FIG. 4D shows representative phase-contrast images of Matrigel-cultured Sca-1^{pos} CD45^{neg} Pecam^{neg} cells (S+45−P−), total lung, AT2 cells, and Sca-1^{pos} CD45^{neg} Pecam^{neg} CD34^{neg} cells (S+45−P−34+). FIGS. 4E-4T show cultures grown on Matrigel subjected to IF for SP-C (green) and CCA (red) (FIGS. 4E, 4G, 4I, 4K, 4M-4T) or SP-C (green) and AQ5 (red) (FIGS. 4F, 4H, 4J, 4L) and analyzed by deconvolution microscopy to assess differentiation. All scale bars, 10 μm. Images represent similar results from at least three independent experiments. FIGS. 4E-4L show that BASC cultures contained SP-C^{pos} CCA^{neg} cells (arrows in FIGS. 4E and 4G, enlarged in FIGS. 4I and 4K), AQ5^{pos} cells (arrows in FIG. 4F, enlarged in FIG. 4J), and SP-C^{neg} CCA^{pos} cells (arrowheads in FIG. 4G, enlarged in FIG. 4K). Only SP-C^{pos} cells were present in AT2 cell cultures (FIG. 4H, enlarged in FIG. 4L). FIGS. 4M-4P show differentiated cells derived from a single Sca-1^{pos} CD45^{neg} Pecam^{neg} CD34^{pos} cells were SP-C^{pos} CCA^{neg} (arrows) (FIGS. 4M and 4O) or SP-C^{neg} CCA^{pos} cells (arrowheads) (FIG. 4N and 4P). Cells in FIGS. 4M and 4N were derived from a single cell, and cells in FIGS. 4O and 4P were derived from another single cell. FIGS. 4Q-4T show single cells on the periphery of clusters derived from fifth (FIGS. 4Q and 4R) or eighth FIG. 4S and 4T passage BASCs were SP-C^{pos} CCA^{neg} (arrows) (FIGS. 4Q and 4T) or SP-C^{neg} CCA^{pos} cells (arrowheads) (FIGS. 4R and 4S).

FIGS. 5A-5E show BASC expansion in adenocarcinoma precursors and tumors. All bar graphs show mean±SD. FIGS. 5A and 5B show IF for CCA and SP-C as in other figures, from wild-type (FIG. 5A) and Lox-K-ras (FIG. 5B) mice 1 week after AdCre. Expanded numbers of BASCs (arrows) are observed in Lox-K-ras lung. Images, 400×; scale bars, 10 μm. FIG. 5C shows distribution of BASC number at time points indicated after AdCre. The percentage of TBs containing one to eight BASCs is shown for wild-type (+/+, left) and Lox-K-ras (K/+, right) lungs. FIG. 5D show the percentage of Lox-K-ras TBs with at least one BASC detected for each AdCre dose (particle-forming units/ml). FIG. 5E shows the percentage of TBs with at least one BASC in Lox-K-ras mice (K/+) compared to wild-type mice (+/+) is shown for each condition.

FIGS. 6A-6C show the specific effect on BASC cultures after K-ras G12D activation. FIG. 6A shows the fold change in the number of cells (mean±SD) present after culture and infection with Adeno Empty (AdE) or Adeno Cre (AdCre) for LSL-K-ras G12D BASC and AT2 cell cultures. Cell number was determined by FACS. FIG. 6B shows the fold change in the percentage of the total population (AdCre/AdE) represented by BASCs, AT2 cells, and Clara/unidentified cells, determined by FACS (mean±SD). FIG. 6C shows the results of PCR to assess recombination in infected BASC or AT2 cell cultures. wt, product amplified from wild-type allele; one-lox, product from recombined Lox-K-ras allele; + control, DNA from a Lox-K-ras tumor.

FIGS. 7A and 7B show cooperation between naphthalene treatment and K-ras tumorigenesis in vivo. Tumor number (FIG. 7A) and tumor area (FIG. 7B) (calculated as the ratio of tumor area to lung area) in Lox-K-ras mice (K/+) after treatment with naphthalene prior to infection (Nap Ad Cre), compared to controls treated with corn oil only (Oil Ad Cre), are shown. Data are the mean±SD from one of three independent experiments that had similar results.

FIG. 8 is a diagram showing different types of lung cells.

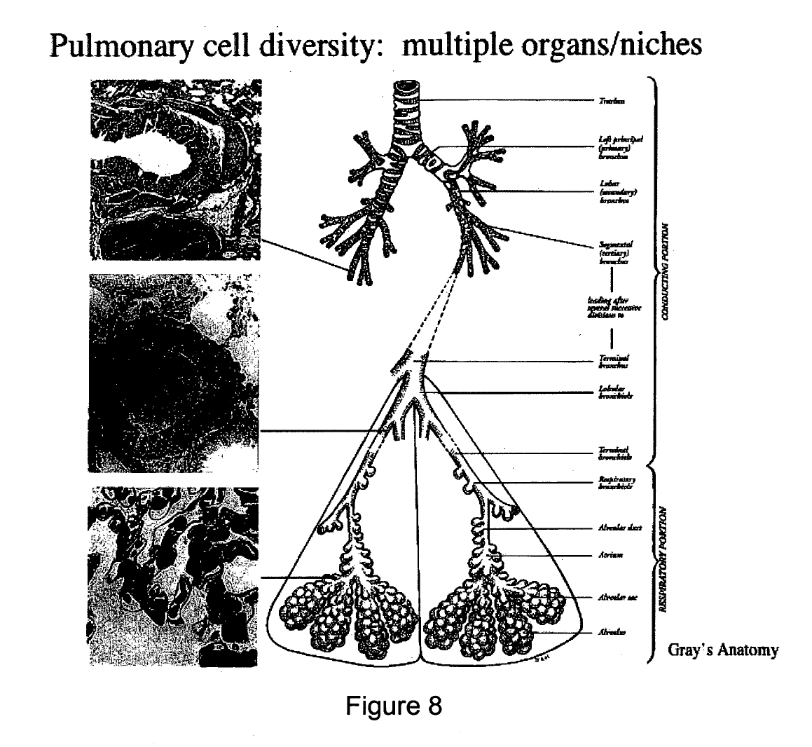

FIG. 9 is a diagram showing the K-ras mouse.

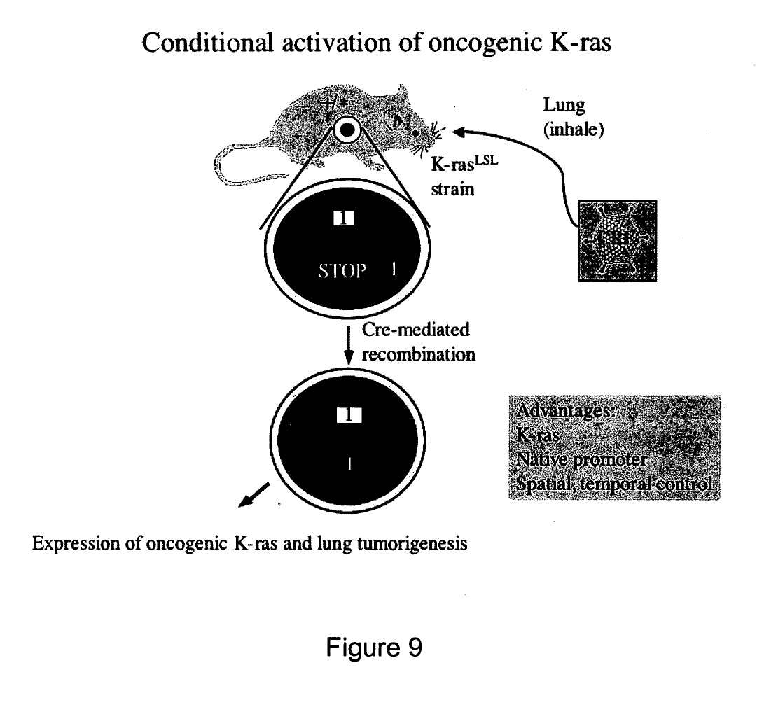

FIG. 10 is a set of images showing the course of tumor progression.

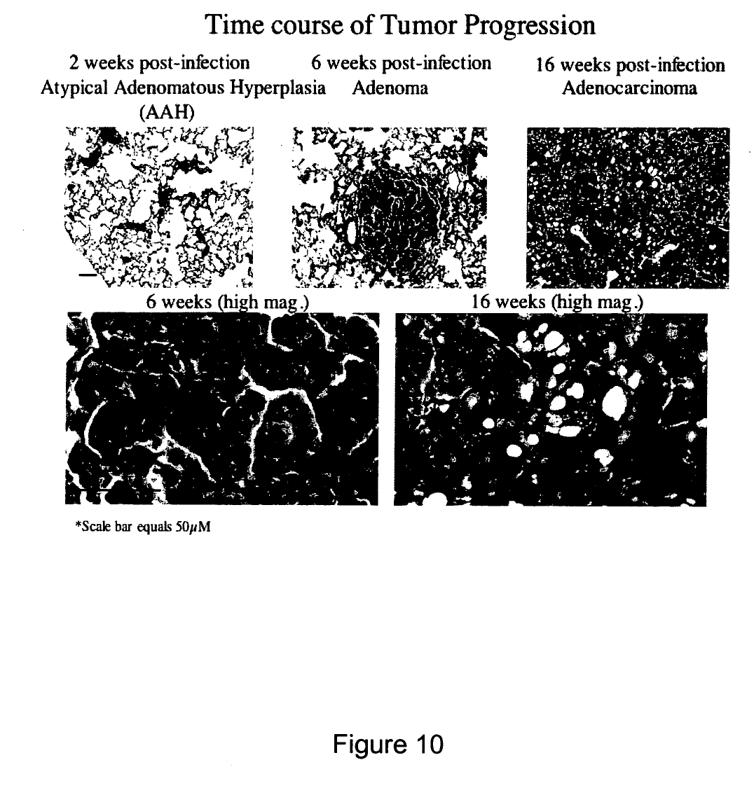

FIG. 11 is a schematic diagram showing epithelia variety in proximal to distal lung.

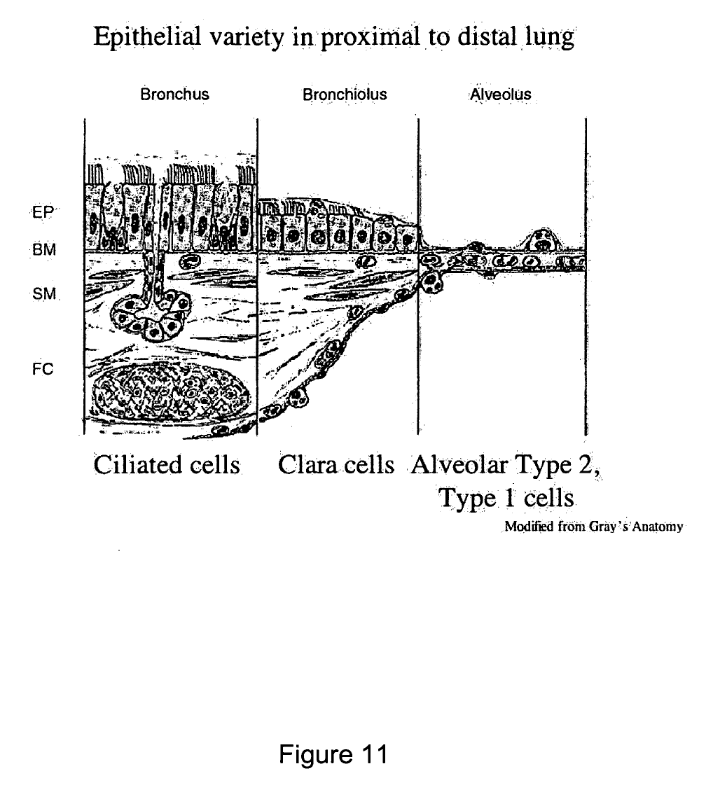

FIG. 12 is a set of images showing adenomas positive for Pro Surfactant Protein C.

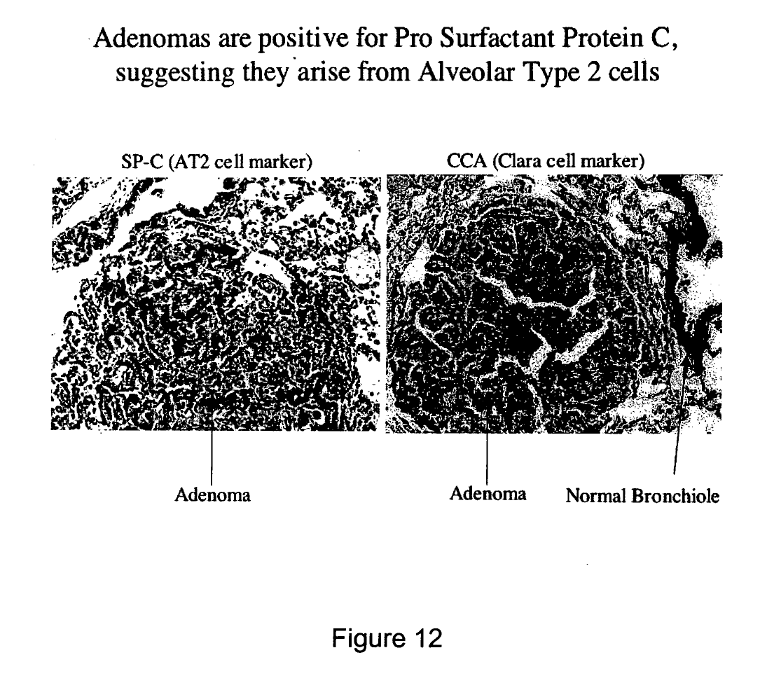

FIG. 13 is an image showing SP-C+ cells present in clusters in K-ras tumors.

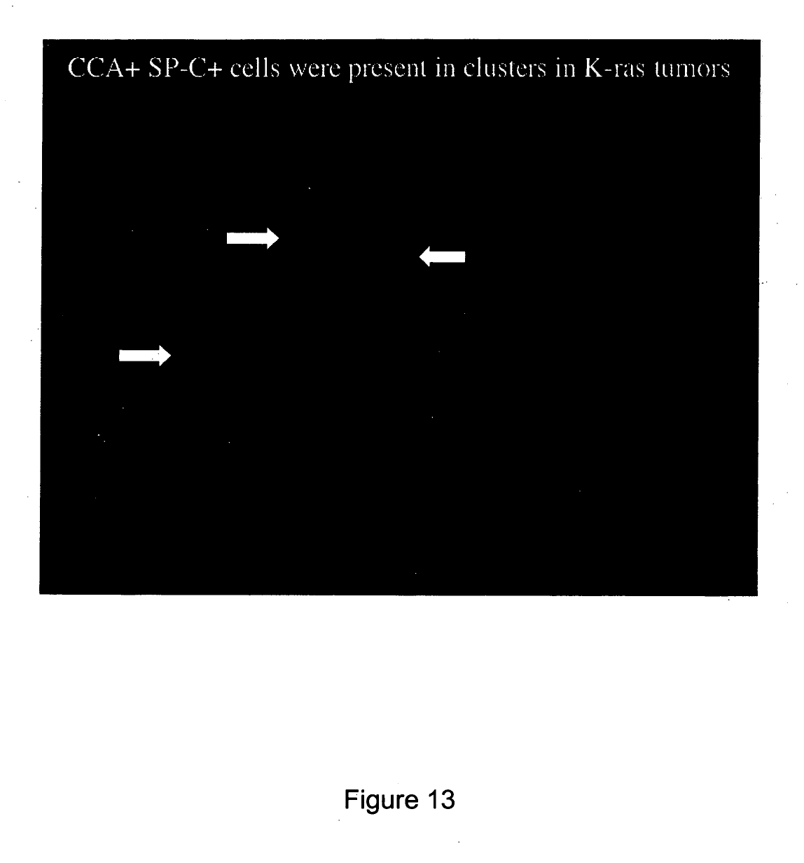

FIG. 14 is a set of images showing adenomas appear to aris near terminal bronchioles.

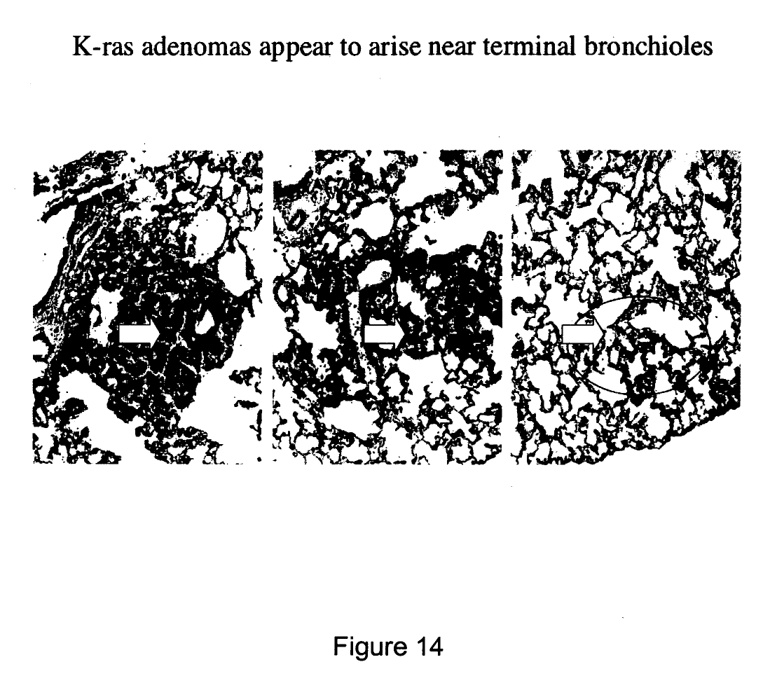

FIGS. 15A and 15B show FACS analysis of BASC changes in vivo confirms IF analyses. FIG. 15A shows the % of the total represented by Sca-1^{pos} CD45^{neg} Pecam^{neg} cells in naphthalene-treated mice was divided by the % in corn oil control-treated mice to obtain the fold change after treatment. Data are the average from three mice. FIG. 15B shows the % of the total represented by Sca-1^{pos} CD45^{neg} Pecam^{neg} cells in wild-type (+/+) and Lox-K-ras (K/+) in AdCre-infected mice was divided by the % in AdE-infected mice to obtain the fold change after treatment for BASCs. Similarly, the % of the total represented by Sca-1^{neg} CD45^{neg Pecam}^{neg} autofl^{hi} cells was compared for AT2 cells. Data are from 3-4 mice five days after infection.

FIG. 16 shows IF analysis of colonies arising from feeder-grown cultures. SP-C, red; CCA, green; DAPI, blue. Note that small, rounded nuclei are in epithelial cells, whereas larger, more oblong nuclei correspond to feeder cells that do not stain for SP-C or CCA. BASC colonies are entirely composed of SP-C+, CCA+ cells. Total lung colonies contain some SP-C+, CCA+ cells, and other total lung colonies contain only SP-C+ cells (right image). AT2 cell colonies are solely SP-C+.

FIGS. 17A-17G show a mixing experiment demonstrating the clonal nature of BASC colonies. Various ratios of GFP+ BASCs, AT2 cells, or total unsorted cells from X-linked GFP mice and GFP- cells from wild-type mice were mixed together and cultured for one week on feeders. The composition of each resulting colony was assessed by fluorescent microscopy. FIGS. 17A and 17C show phase-contrast images of BASC colonies. FIGS. 17B and 17D show epifluorescence of same colonies. Both colonies arose in cultures containing a 1:1 mixture of GFP+:GFP− cells. FIGS. 17A and 17B show an entirely GFP+ colony; FIGS. 17C and 17D show an entirely GFP− colony. FIGS. 17E and 17F are images showing a total unsorted lung colony containing both GFP+ and GFP− cells (arrows) that arose from plating a 1:1 mixture of GFP+:GFP− cells. FIG. 17G is a graph showing regression analysis which demonstrates that the % GFP+ cells is directly proportional to GFP+ colony formation only for BASCs.

FIG. 18 shows representative FACS analysis of Matrigel-grown cell cultures as shown in FIG. 16.

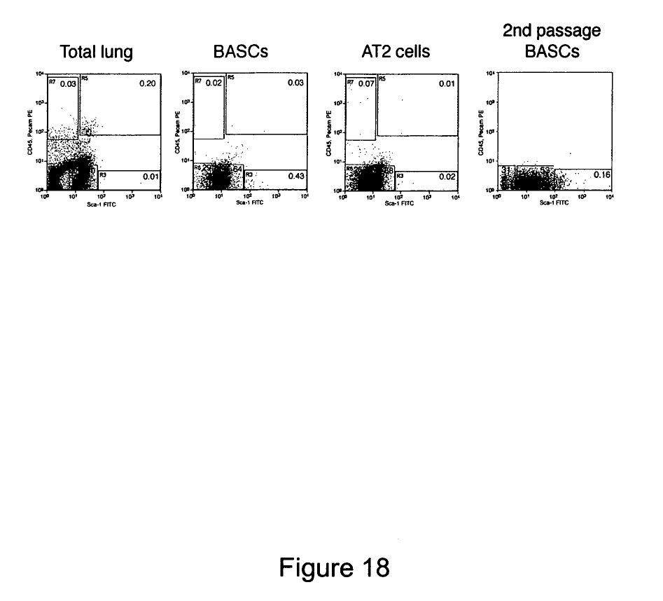

FIGS. 19A-19L show IF examining surface markers used for FACS isolation. FIGS. 19A-19C show spleen (FIGS. 19A, 19B, for positive control) and lung (FIG. 19C) sections stained with secondary antibody only (FIG. 19A), for CD45 (FIG. 19B, green), or CD45 and CD34 (green and red, respectively)(FIG. 19C). Importantly, the CD34-positive cells in TBs (arrow) were CD45-negative (FIG. 19C). Insets show enlargements. Asterisk indicates autofluorescent cells. In FIGS. 19D, 19E (enlargement of FIG. 19D), and 19F, lung sections stained for CD34 (green) and CCA (red) are shown. Thick arrows indicate CD34-positive, CCA-negative endothelial cells; thin arrows indicate CD34-positive, CCA-positive cells at BADJ. The asterisk indicates an example of an autofluorescent signal. FIGS. 19G-19I show lung sections stained for Sca-1 (green) and CCA (red). In FIG. 19G, the arrowhead points to an example of Sca-1 staining in a ciliated cell in a bronchiole; the thick arrow shows Sca-1 staining in endothelial cells. In FIGS. 19H and 19I (enlargement of 19H), the arrow points to a Sca-1- and CCA-positive cell at the BADJ in a TB. In FIG. 19J, Sca-1 (red) and CD34 (green) dual IF are shown. The arrow indicates two columnar cells positive for CD34 and Sca-1 at the BADJ. The thick arrow indicates CD34 and Sca-1 staining in endothelial cells. The asterisk indicates an autofluorescent red blood cells within a vessel. FIG. 19K shows an image created from four color IF for CCA (diffuse red signal), Sca-1 (punctate, brighter red signal), CD34 (green) and DAPI (blue). The arrow indicates a cell at the BADJ positive for CCA, CD34, and Sca-1. CD34 and Sca-1 staining reveal that only the right surface of the cell is detected in the section. Yellow areas represent overlap between Sca-1 and CD34 staining. FIG. 19L shows merged CCA and Sca-1 signals only from image in FIG. 19K.

### DETAILED DESCRIPTION

Injury models have suggested that the lung contains anatomically and functionally distinct epithelial stem cell populations. In the present invention, such a regional pulmonary stem cell population, termed bronchioalveolar stem cells (BASCs) has been isolated. Identified at the bronchioalveolar duct junction (BADJ), BASCs are resistant to bronchiolar and alveolar damage and proliferate during epithelial cell renewal in vivo. BASCs exhibit self-renewal and are multipotent in clonal assays, highlighting their stem cell properties. Furthermore, BASCs expand in response to oncogenic K-ras in culture and in precursors of lung tumors in vivo. These data indicate that BASCs are a stem cell population that maintains the bronchiolar Clara cells and alveolar cells of the distal lung and that their transformed counterparts give rise to adenocarcinoma. Although bronchiolar cells and alveolar cells are proposed to be the precursor cells of adenocarcinoma, this invention points to BASCs as the cells of origin for this subtype of lung cancer.

The pulmonary system contains a variety of epithelial cell populations that each reside in distinct anatomical locations. Basal, secretory, and ciliated cells line the trachea and the proximal conducting airways; neuroendocrine cells are present in small numbers. The nonciliated, columnar Clara cells comprise the majority of the bronchiolar and terminal bronchiolar epithelium in mice, and alveolar type I (AT1) and type II (AT2) cells constitute the alveolar epithelium (Bannister, L. H. (1999) Gray's Anatomy, Williams et al. eds. (New York: Churchill Livingstone), 1666-1672).

Injury models have suggested that functionally distinct epithelial stem cell populations exist in precise anatomical locations in the lung (Otto, W. R. (2002) J. Pathol. 197, 527-535). A putative tracheal and proximal conducting airway stem cell niche has been identified by resistance to the detergent polidocanol and sulfur dioxide (Borthwick et al. (2001) Am. J. Respir. Cell Mol. Biol. 24, 662-670). Bleomycin administration results in AT1 cell injury, and it has been proposed that AT2 cells repair the damaged alveolar epithelium (Aso et al. (1976) Lab. Invest. 35, 558-568). Naphthalene, a pollutant that specifically ablates Clara cells, has been used to identify two distinct stem cell niches. In proximal airways, naphthalene-resistant Clara cells are closely associated with neuroendocrine bodies, whereas resistant cells are found at the junction between the conducting and respiratory epithelium (the bronchioalveolar duct junction, or BADJ) in terminal bronchioles (TBs) (Giangreco et al. (2002) Am. J. Pathol. 161, 173-182; Hong et al. (2001) Am. J. Respir. Cell Mol. Biol. 24, 671-681; Reynolds et al. (2000) Am. J. Pathol. 156, 269-278; Reynolds et al. (2000) Am. J. Physiol. Lung Cell. Mol. Physiol. 278, 1-1256-1-1263). “Side population” cells exhibiting Hoechst dye efflux properties similar to those of hematopoietic stem cells have been isolated from lung (Giangreco et al. (2003) Am. J. Physiol. Lung Cell. Mol. Physiol. 286, L624-L630; Goodell et al. (1996) J. Exp. Med. 183, 1797-1806; Summer et al. (2003) Am. J. Physiol. Lung Cell. Mol. Physiol. 285, L97-L104). Despite these advances, methods to isolate cells from putative stem cell niches in the lung and functional tests for such populations have not been reported prior to the present invention.

Previously, understanding of lung stem cell biology was limited. The identity of the cell of origin in lung tumorigenesis was also largely unknown. This was due in part to the predominantly advanced stage of disease in most patients at the time of diagnosis (Minna et al. (2002) Cancer Cell 1, 49-52). Interestingly, many of the subtypes of non-small cell lung carcinoma share characteristics with differentiated cells found in the distinct locations in which the tumors arise. For example, squamous cell carcinomas exhibit keratinization like mature epithelial cells in the trachea and proximal airways and generally arise in bronchi, whereas most adenocarcinomas display Clara or AT2 cell markers and are generally peripheral or endobronchial (Rosai et al. (1995) In Atlas of Tumor Pathology (Washington, D.C.: Armed Forces Institute of Pathology)). Prior to the present invention, whether the cancers arise from these differentiated cell compartments or from stem or progenitor cells was not known.

A previously developed “Lox-Stop-Lox” K-ras conditional mouse strain (referred to as LSL-K-ras G12D), in which expression of oncogenic K-ras is spatially and temporally controlled by a removable transcriptional termination (“stop”) element (Jackson et al. (2001) Genes Dev. 15, 3243-3248) was used as described below. Intranasal infection with recombinant adenoviral Cre (AdCre) results in deletion of the stop element, producing the Lox-K-ras allele that expresses K-ras G12D. Lox-K-ras mice develop atypical adenomatous hyperplasias (AAH; hyperproliferations of AT2 cells) that appear to progress to adenomas and then overt adenocarcinomas (Jackson et al., supra). This progression, together with the observation that Lox-K-ras adenocarcinomas were positive for the AT2 cell-specific marker prosurfactant apoprotein-C (SP-C), was consistent with previous studies that implicated AT2 cells as the target cells in rodent and human lung adenocarcinomas. However, evidence in other murine models and human specimens points to Clara cells as the cell of origin of adenocarcinoma (Dermer, G. B. (1982) Cancer 49, 881-887; Gunning et al. (1991) Exp. Lung Res. 17, 255-261; Mason et al. (2000) Am. J. Pathol. 156,175-182; Mori et al. (1993) Cancer 72, 2331-2340; Mori et al. (2001) Mod. Pathol. 14, 72-84; Thaete et al. (1991) Exp. Lung Res. 17, 219-228; Wikenheiser et al. (1992) Cancer Res. 52, 5342-5352).

During analysis of Lox-K-ras tumors, we identified a novel cell type, termed double-positive cells (DPCs). DPCs expressing both SP-C and the Clara cell-specific marker CCA (also known as CC10 or CCSP) were found in adenomas, particularly in lesions continuous with bronchiolar hyperplasia (Jackson et al., supra). We now show that counterparts of the SP-C^{pos} CCA^{pos} Lox-K-ras DPCs, renamed BASCs, exist in normal lung, function in lung homeostasis, and possess characteristics of regional stem cells. Furthermore, we provide evidence that BASCs are the cells of origin of adenocarcinoma.

BASCs are a cell population in the adult mouse lung that has properties of stem cells and is linked to lung tumor initiation. Wild-type BASCs are found in a putative stem cell niche, proliferate during epithelial repair in vivo, and are capable of multipotent differentiation and self-renewal in culture. BASCs may be a regional stem cell population in the distal lung. Increases in BASC number are observed in Lox-K-ras mice with AAH and adenomas. The temporal and spatial correlation of BASC expansion with early-stage lesions suggests that AAH arise from transformed BASCs. Stimulation of BASCs by airway injury resulted in an increase in tumor number and size, further implicating BASCs in tumorigenesis.

Defining Lung Stem Cells

BASCs are stem cells that share characteristics with previously defined adult stem cell populations (Blanpain et al. (2004) Cell 118, 635-648; Chiasson et al. (1999) J. Neurosci. 19, 4462-4471; Doetsch et al. (1999) Cell 97, 703-716; Johansson et al. (1999) Cell 96, 25-34; Kruger et al. (2002) Neuron 35, 657-669; Morris et al. (2004) Nat. Biotechnol. 22, 411-417; Tropepe et al. (2000) Science 287, 2032-2036). Importantly, cultured BASCs exhibit proliferative capacity and self-renewal and are multipotent in clonal assays. BASCs retain these properties over eight passages to date.

With the identification and purification of BASCs, it is now possible to determine the ability of BASCs to produce differentiated lung epithelia and self-renew in vivo to define their functions further. There is an assay for reconstitution of denuded trachea subcutaneously implanted into immunodeficient mice, and this system provides a tracheal-like environment (Delplanque et al. (2000) J. Cell Sci. 113, 767-778). Bone-marrow cells may contribute to lung tissue after damage (Harris et al. (2004) Science 305, 90-93; Kotton et al. (2001) Development 128, 5181-5188; Krause et al. (2001) Cell 105, 369-377), but it is unlikely that stem cells normally residing in the lung can survive conditions in circulation and home properly to the BADJ. Therefore, experiments to test stem cell function of BASCs may be done by introduction of the cells into their original niche. This may be done, optionally, by introduction of genetically marked BASCs into the respiratory system of wild-type mice after bronchiolar or alveolar cell damage. Alternatively, it would be informative to genetically label endogenous BASCs or other epithelial cells specifically in vivo after lung damage and track their progeny without introduction of exogenous cells.

Current evidence supports the existence of multiple stem cell niches in the lung (Otto, W. R. (2002) J. Pathol. 197, 527-535). Our work indicates that BASCs are a stem cell population for distal lung epithelia with potentiality limited to Clara, AT2, and AT1 cells. Their localization to the BADJ places BASCs next to each of the niches in which their putative progeny reside (the terminal bronchiole for Clara cells and the alveolar space for AT2 cells). Furthermore, BASCs are normally in a quiescent state and can be activated in response to bronchiolar and alveolar injury in vivo.

The expansion observed in BASCs during airway renewal was subtle, leading us to hypothesize that BASCs give rise to a progenitor population, perhaps a subtype of Clara cells, that restores damaged terminal bronchiolar epithelium. BASCs were the first cells to proliferate in response to naphthalene, and they comprised the majority of the proliferating population up to one week after damage. BrdU^{pos} Clara cells were situated next to Brd^{neg} BASCs at later stages of terminal bronchiolar repair, consistent with the hypothesis that they arose from BASCs.

BASCs may also give rise to an alveolar progenitor population to maintain alveolar homeostasis in vivo, given their response to bleomycin treatment. Although the commonly held view has been that AT2 cells are progenitors for AT1 cells because cultured AT2 cells acquire AT1-like properties in culture (Isakson et al. (2001) Am. J. Physiol. Cell Physiol. 281, C1291-C1299), the work presented herein suggests that BASCs give rise to AT1 cells. One possibility is that BASCs give rise to AT2 cells, which, in turn, produce AT1 cells. The results of Aso et al. suggest that there may be two niches that mediate alveolar repair; both proliferation of AT2 cells in alveolar spaces and expansion and differentiation of some bronchiolar cells was observed after bleomycin-mediated damage (Aso et al. (1976) Lab. Invest. 35, 558-568). Furthermore, Daly et al. found that SP-C and CC10 coexpression was present in a subset of distal bronchiolar cells following bleomycin-mediated lung injury and proposed that this finding may be evidence for a stem cell population in bronchioles that responds to alveolar damage nearby (Daly et al. (1997) Toxicol. Appl. Pharmacol. 142, 303-310; Daly et al. (1998) Lab. Invest. 78, 393-400).

Elucidating Lung Cancer Cells of Origin

This work provides the first evidence that stem cells are the target cell population in lung adenocarcinoma. Prior work from Dick, Weissman, and colleagues has provided compelling evidence that stem cells can be the target cells in tumorigenesis for hematopoietic malignancies (Bonnet et al. (1997) Nat. Med. 3, 730-737; Cozzio et al. (2003) Genes Dev. 17, 3029-3035; Passegue et al. (2004) Cell 119, 431-443). Importantly, BASCs are expanded at early stages of tumorigenesis in vivo and exhibited the first proliferative response following K-ras G12D activation in culture. We have also observed BASC expansion in association with lung tumorigenesis initiated by expression of a point mutant p53 allele (Olive et al. (2004) Cell 119, 847-860), ruling out a specific effect of AdCre on BASC expansion (data not shown). Since Clara cells or AT2 cells are widely thought to be the precursor cells in adenocarcinoma, this work points to an alternative model of the cell of origin for this subtype of lung cancer. There are an expanded number of BASCs in early lesions. It is conceivable that a progenitor cell population that gains the property of self-renewal may be equally capable of supporting tumor initiation (Cozzio et al. (2003) Genes Dev. 17, 3029-3035).

Lung tumors may arise from expansion of stem cells and that advanced tumors retain characteristics of differentiated lineage(s) due to influences from the microenvironment or continued oncogenic signaling. For example, expansion of BASCs stimulated by oncogenic K-ras and continued proliferation in the context of constitutive K-ras signaling may result in adenocarcinomas largely composed of AT2 cells. The analysis presented herein of the effect of activated K-ras on BASC differentiation in culture supports this reasoning. Further, most cells in Lox-K-ras tumors are SP-C^{pos} CCA^{neg}, and some tumor cells are also positive for AQ5 (Jackson et al. (2001) Genes Dev. 15, 3243-3248; data not shown); the immunophenotype of Lox-K-ras tumors matches the alveolar differentiation pattern observed for BASCs. It is likely that a corresponding human BASC population exists, and it too plays a role in adenocarcinoma development, and is consistent with adenocarcinomas most frequently resembling bronchiolar and alveolar components of normal human lung (Rosai et al. (1995). In Atlas of Tumor Pathology (Washington, D.C.: Armed Forces Institute of Pathology)).

Identification of the BASC population, its niche, and a means to isolate and propagate this population provides a better understanding of the cellular and molecular mechanisms of lung development, homeostasis, and disease. Chronic lung diseases may be alleviated by directed differentiation of lung stem cells to restore defective lung epithelia, for example, through the use of compounds identified using the methods described herein. These cells may also be used for ex vivo gene therapy of genetic diseases affecting the lung such as cystic fibrosis. Regarding lung cancer, BASCs were detected in established tumors, suggesting that they may contribute continuously to tumor development and progression. Tumor-associated BASCs may constitute a tumor stem cell population similar to those that have been defined in breast and brain cancers (Al-Hajj et al. (2003) Proc. Natl. Acad. Sci. USA 100, 3983-3988.; Reya et al. (2001) Nature 414, 105-111; Singh et al. (2003) Cancer Res. 63, 5821-5828; Singh et al. (2004) Nature 432, 396-401) and, as such, may be a critical target for anticancer therapy. Finally, additional studies pointing to a role for BASCs in tumor initiation may fuel more effective early-identification markers and chemoprevention strategies for lung cancer by specifically targeting hyperproliferative BASCs in the early stage of disease.

The following examples are meant to illustrate the invention and should not be construed as limiting.

### EXAMPLE 1

### Isolation of BASC cells

In the present invention, a method of isolating BASCs has been developed. Based on their staining for AT2 and Clara cell-specific markers, their response to damage in vivo, and their location at the bifurcation between bronchiolar and respiratory epithelium, BASCs may constitute a stem or progenitor cell population that maintains Clara cells and AT2 cells in adult lung. As CCA and SP-C are cytoplasmic, intracellular markers, they are less preferable for live cell isolation of BASCs by FACS. A preferable marker is a be marker that is present on the surface stem cells. Sca-1, for example, is present on the surface of hematopoietic stem cells and mammary-gland progenitors (Morrison et al. (1994) Immunity 1, 661-673 and references within; Welm et al. (2002) Dev. Biol. 245, 42-56), and is useful in the present method as a marker for selecting BASC cells.

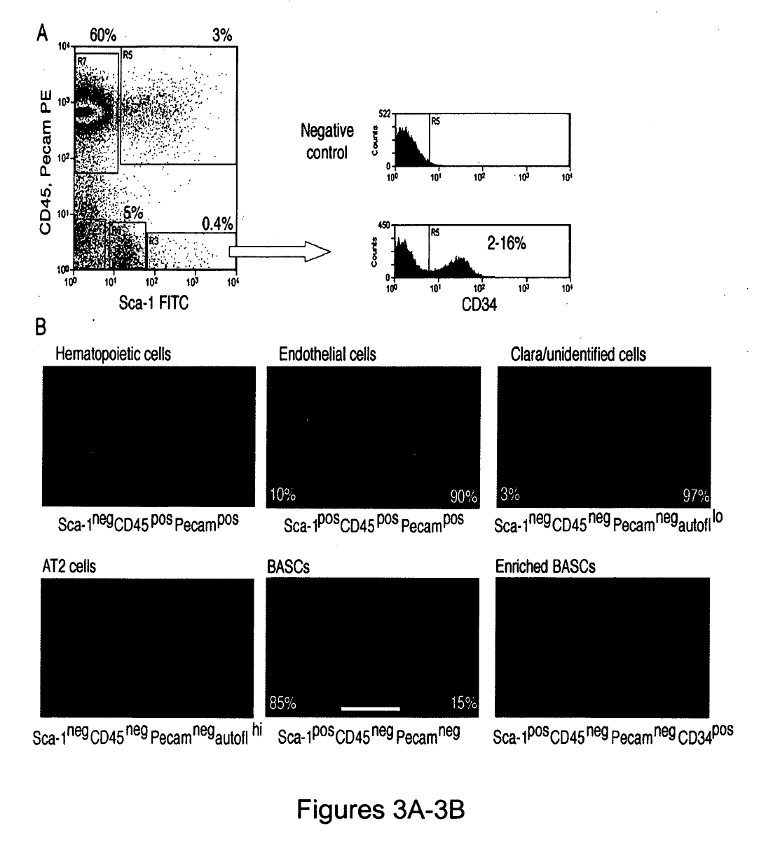

The following protocol was developed for the purification of BASCs by flourescence-activated cell sorting (FACS), a method standard in the art and described in more detail below. As is known in the art, any number of cellular markers may be used for this protocol. Fresh lung cells are prepared, for example, as described herein, red blood cells may be removed by lysis, and the hematopoietic and endothelial lineages are excluded, using markers specific for these cells, for example, CD45 and Pecam, respectively. From the remaining cells, Sca-1^{pos} cells can be sorted to determine if they contained the BASC population (FIG. 3). This population represented 0.4% of the total lung cell preparation in 4- to 8-week-old wild-type mice (FIG. 3A). Immunophenotyping of the FACS-sorted populations using the CCA and SP-C markers demonstrates that only the Sca-1^{pos} CD45^{neg} Pecam^{neg} population contain BASCs (FIG. 3B). Eighty-five percent of these cells are CCA^{pos} SP-C^{pos} cells, whereas 15% are CCA^{pos} ciliated cells. The Sca-1^{neg} CD45^{neg} Pecam^{neg} population with high autofluorescence comprises SP-C^{pos} CCA^{neg} AT2 cells and represents 5%-20% of the total lung population. The Sca-1^{neg} CD45^{neg} Pecam^{neg} population with low autofluorescence contains CCA^{pos} Clara cells and other unidentified cells. The Sca-1^{neg} CD45^{pos} Pecam^{pos} population contained hematopoietic cells, including macrophages. The Sca-1^{pos} CD45^{pos} Pecam^{pos} population contains mostly endothelial cells, and 10% contained highly autofluorescent, Pecam^{neg} Sca-1^{neg} AT2 cells (data not shown).

BASCs may be purified further based on positive staining for stem cell markers, for example, CD34, a known marker of skin epithelial and hematopoietic stem cells (Blanpain et al. (2004) Cell 118, 635-648; Ramalho-Santos et al. (2002) Science 298, 597-600; Ivanova et al. (2002) Science 298, 601-604; Tumbar et al. (2004) Science 303, 359-363; Morris et al. (2004) Nat. Biotechnol. 22, 411-417). Two to sixteen percent of Sca-1^{pos} CD45^{neg} Pecam^{neg} cells are positive for CD34 (FIG. 3A). Whereas the Sca1^{pos} CD45^{neg} Pecam^{neg} CD34^{neg} population contain ciliated cells and CCA^{pos} SP-C^{pos} cells (data not shown), all cells in the Sea-1^{pos} CD45^{neg} Pecam^{neg} CD34^{pos} population are positive for CCA and SP-C, and no ciliated cells were present (FIG. 3B). Thus using a stem cell marker to separate cells by FACS, the Sca-1^{pos} CD45^{neg} Pecam^{neg} CD34^{pos} population is further enriched for BASCs.

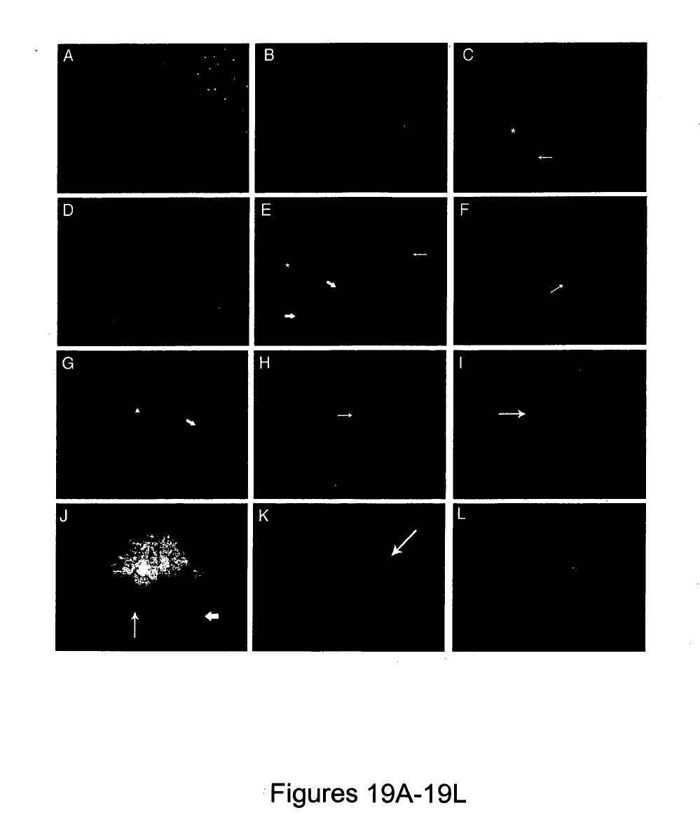

To verify the anatomical location of the cells selected using this FACS isolation scheme, IF for CCA, Sca-1, and CD34 can be performed on lung tissue sections. Sca-1 and CD34 are detected in endothelial cells in capillaries and larger blood vessels. All Sca-1^{pos} cells in bronchioles were CD34^{neg} ciliated cells, whereas Sca1^{pos} cells in terminal bronchioles were at the BADJ and were CCA^{pos} (data not shown). CD34 staining is detected in some Clara cells in bronchioles (FIG. 19). Importantly, Sca-1^{pos} CD34^{pos} CCA^{pos} cells are located exclusively at the BADJ (FIG. 19). To determine if these cells were of hematopoietic origin, sections are also stained for the marker CD45. The CD34^{pos} cells are negative for CD45 (FIG. 19). Thus, the use of Sca-1- and CD34-positive surface staining, together with exclusion of hematopoietic and endothelial lineage, allows for the isolation of the same BADJ cells identified by IF for CCA and SP-C. Together, these results provided a FACS methodology sufficient to isolate live lung cell populations and compare their ability to self-renew and differentiate in culture.

Preparation of Lung Tissue

Mice were anesthetized with avertin, perfused with 10 mL PBS, followed by intra-tracheal instillation of 1 mL dispase (Becton Dickinson, 50 U/mL) and 1 mL 1% LMP agarose. Lungs were iced, minced and incubated in 0.001% DNAse (Sigma) and 2 μg/mL collagenase/dispase (Roche) in PBS for 45 min at 37° C., filtered through 100 μm and 40 μm cell strainers (Fisher), and centrifuged at 800 rpm, 5 min at 4° C. Cells were resuspended in red blood cell lysis buffer (0.15 M NH₄Cl, 10 mM KHCO₃, 0.1 mM EDTA) for 4 min, washed in DME/10% fetal bovine serum (FBS), resuspended in PBS/10% FBS (PF10) at 1 million/100 μL and incubated 15 min at 4° C. with 1 μL of the appropriate antibody for surface marker staining.

FACS Analysis

Single-cell suspensions from lung were prepared as described above, using a procedure from Bortnick et al. (2003) Am. J. Physiol. Lung Cell. Mol. Physiol. 285, L869-L878 with modifications. Sca-1-FITC, CD45.1-Biotin, Pecam-Biotin, CD34-PE, and Streptavidin-PE were from Pharmingen. 7-AAD (Molecular Probes) staining eliminated dead cells. Cell sorting was performed with a Cytomation MoFlo (DakoCytomation; Carpinteria, Calif.), and data were analyzed with Cytomation Summit software.

### EXAMPLE 2

### BASCs Exhibit Stem Cell Properties in Culture

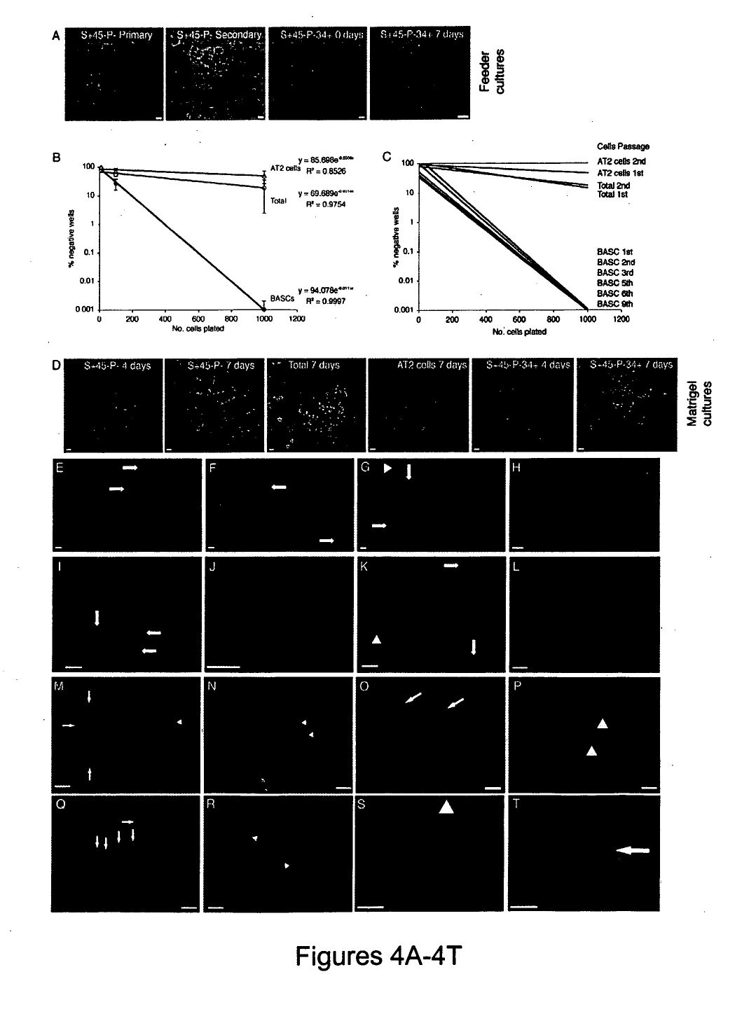

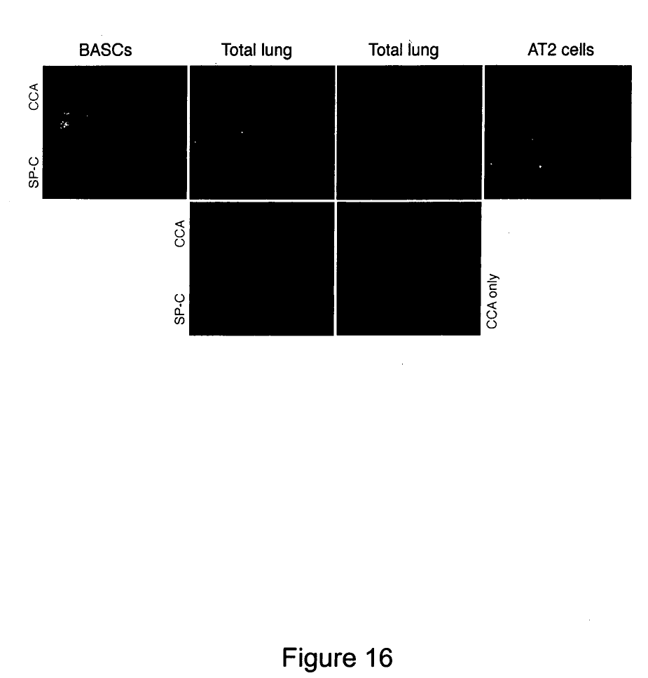

Lung epithelial cells harvested using the above-described FACS protocol formed colonies after one week when grown on feeders (FIG. 4A). All cells in feeder-grown colonies from BASC cultures were CCA^{pos SP-C}^{pos} suggesting that growth on feeders maintained the cells' undifferentiated state. Eighty percent of colonies on feeders from total lung cultures also contained CCA^{pos} SP-CPO5 cells, but in all colonies examined, these cells were only a portion of the colony. The remainder of total lung colonies contained CCA^{neg} SP-C^{pos} cells. Thus, the colonies present in total lung cultures likely arose from a mixture of AT2 cells, BASCs, and other unidentified cells, an important note for subsequent analyses of total lung cultures. As expected, AT2 cultures only contained SP-C^{pos} cells (FIG. 16).

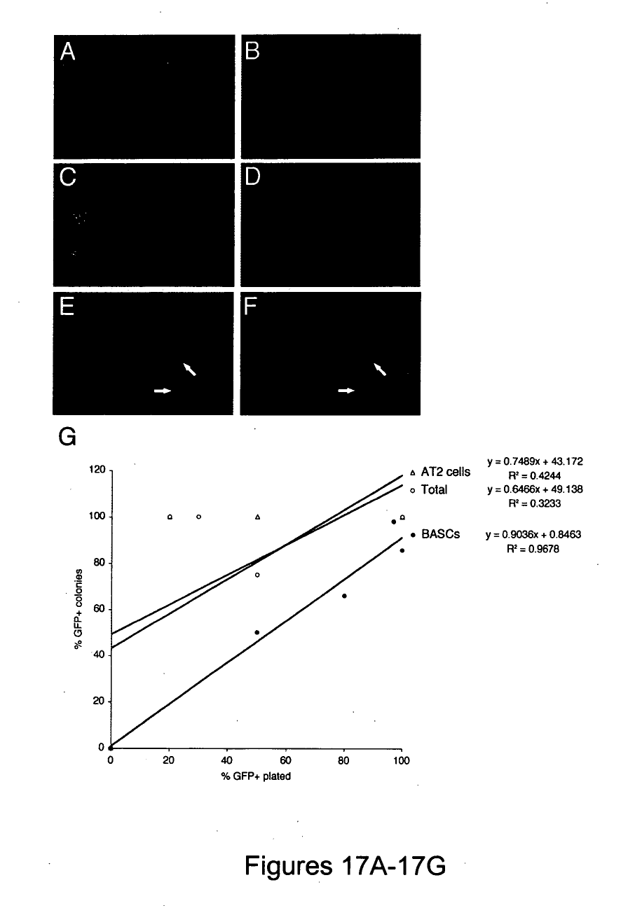

Several methods were used to show that the colonies arising from BASC cultures were clonal. First, 5% of single-cell cultures of Sca-1^{pos} CD45^{neg Pecam}^{neg} CD34^{pos} cells plated on feeders formed epithelial colonies identical to those arising from multiple Sca-1^{pos} CD45^{neg} Pecam^{neg} cells (FIG. 4A). Plating of neither single AT2 cells nor single total lung cells produced colonies. Second, limiting dilution analysis revealed that the BASC population is 5.5-fold enriched for single cells that can give rise to colonies. There was a linear relationship between the cell density and the number of colonies generated from total lung or BASCs but not those generated from AT2 cells (FIG. 4B). Limiting dilution showed that 1 out of 452 total lung cells gave rise to a colony, whereas the clonal frequency was 1 out of 81 for the Sca-1^{pos} CD45^{neg} Pecam^{neg} population. Finally, plating mixtures of GFP^{pos} BASCs from D4/XEGFP mice, which carry an X-linked GFP transgene (Hadjantonakis et al. (1998) Nat. Genet. 19, 220-222), with GFP^{neg} BASCs from wild-type mice only yielded entirely GFP^{pos} GFP^{neg} colonies (FIG. 17). In contrast, mixtures of GFP^{pos} and GFP^{neg} AT2 cells and total lung cells produced mixed colonies (containing GFP^{pos} and GFP^{neg} cells), suggesting that the majority of their colonies were not clonal; these data are consistent with IF and limiting dilution results (FIG. 17).

In addition to clonal colony formation, only BASCs exhibited extensive self-renewal in culture (FIG. 4C). Single-cell suspensions from individual colonies in primary total lung and BASC cultures formed colonies identical to primary cell colonies (FIG. 4A and data not shown), whereas AT2 cultures did not produce secondary colonies. Total lung cell cultures failed to produce colonies in tertiary cultures, perhaps owing to the use of nonclonal colonies (above). In contrast, the Sca1^{pos} CD45^{neg} Pecam^{neg} population has been passaged up to nine times to date without diminution of colony-forming ability (FIG. 4C and data not shown).

BASCs also had a greater capacity for differentiation than other lung epithelial cells (FIG. 4D). By 4 days on Matrigel, Sca-1^{pos} CD45^{neg} Pecam^{neg} cells formed three-dimensional structures containing a central cluster of ˜400 cells with surrounding cells exhibiting short cytoplasmic extensions. By 7 days, the cytoplasmic extensions were more complex and resembled alveolar epithelium. Total lung cell cultures produced similar structures, whereas AT2 cultures remained in small clusters. Furthermore, Sea-1^{pos} CD45^{neg} Pecam^{neg} CD34^{pos} BASC cultures appeared identical to those isolated without CD34 selection (compare images in FIG. 4D).

Immunophenotyping of Matrigel cell cultures with CCA, SP-C, and aquaporin-5 (AQ5), a marker of AT1 cells (Nielsen et al. (1997) Am. J. Physiol. 273, C1549-C1561), confirmed the multilineage differentiation capacity of BASCs. Seven- to ten-day old total lung cell and BASC cultures contained CCA^{pos} SP-C^{neg} cells (Clara-like cells), SP-C^{pos} CCA^{pos} cells (AT2 -like cells), and AQ5^{pos} cells (AT1-like cells) (FIG. 4). Cells with the most abundant SP-C staining were located around the periphery of the cell clusters in BASC cultures shown in FIGS. 4E and 4I. The cells extending from the core cluster and exhibiting cytoplasmic branching reminiscent of AT1 cells were AQ5^{pos}, as were rare cells on the periphery of the clusters (FIGS. 4F and 4J). Dissociation of the clusters revealed that they contained Clara-like and AT2 -like cells (FIGS. 4G and 4K). FACS further confirmed the differentiation status of cells in Matrigel cultures. BASC cultures contained AT2 cells (25%-84% of the culture), Clara/other unidentified cells (28%-70%), and BASCs (0.36%-3.3%). Total lung cultures consisted of Clara/other unidentified cells, AT2 cells, and endothelial cells but no BASCs. AT2 cell cultures retained the AT2 cell population and did not produce BASCs.

In our analyses, 100% of BASC cultures were multipotent, producing Clara- and AT2 -like cells. In contrast, 25% of total lung cultures were multipotent, 50% produced AT2 -like cells only, and 25% produced Clara-like cells only. AT2 cell cultures did not exhibit differentiation (FIGS. 4H and 4L). Importantly, 100% of colonies derived from single BASCs gave rise to both Clara- and AT2 -like cells on Matrigel (n=6, representing three independent experiments) (FIGS. 4M-4P). Furthermore, even after eight passages, BASC clones produced Clara- and AT2-like cells, showing that they retained differentiation capacity while undergoing self-renewal (FIGS. 4Q-4T). No ciliated cells were detected at any time point of culturing based on morphology and staining with antisera raised against acetylated tubulin (data not shown), suggesting that ciliated cells present in the sorted Sca-1^{pos} CD45^{neg} Pecam^{neg} population (FIG. 3) did not survive culture conditions or contribute to differentiation and other culture characteristics.

Together, data from lung cell cultures demonstrated that BASCs are able to self-renew and possess multilineage differentiation potential.

### EXAMPLE 3

### Involvement of BASCs in Lung Cancer

Increases in BASC population are observed in mouse models of lung cancer. Data presented here indicate that increases in BASC population are an early-stage event in lung cancer, and thus are useful in the early detection and diagnosis of such cancers.

CCA-Positive, SP-C-Positive Cells in Normal Adult Lung

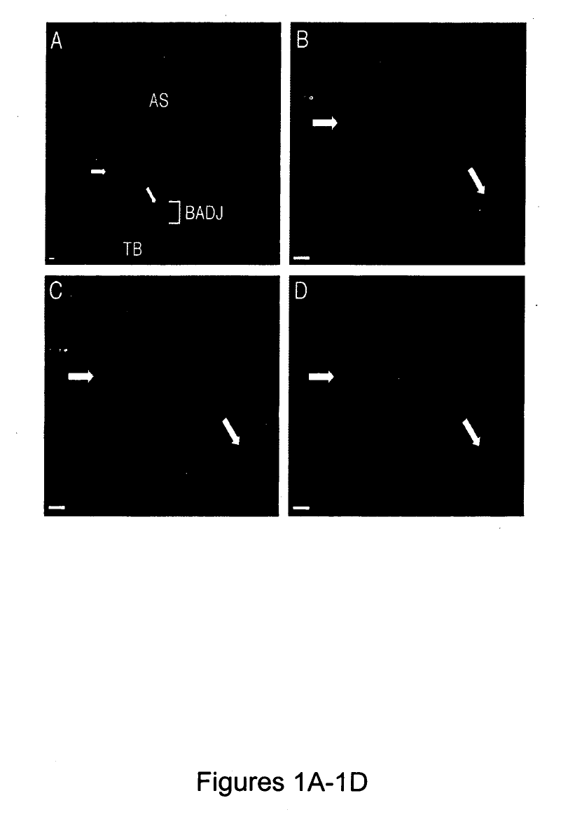

As described above, DPCs (BASCs) are initially observed in small clusters in Lox-K-ras lung tumors, representing a small proportion of the total tumor cell population that largely consists of SPC^{pos} cells (Jackson et al., supra). Tumor DPCs may therefore originate from a cell population in the adult lung similar to embryonic lung epithelial precursors that are positive for Clara and AT2 cell markers (Wuenschell et al. (1996) J. Histochem. Cytochem. 44, 113-123). Therefore, sections of wild-type lung can be examined by dual immunofluorescence (IF) for CCA and SP-C. As shown in FIG. 1, DPCs are identified in normal lung at the BADJ. As expected, CCA staining is present in columnar bronchiolar and terminal bronchiolar cells, whereas SP-C staining is limited to AT2 cells and DPCs. Upon examination of more than 100 terminal bronchioles from five wild-type mice, at least one DPC is detected in 35%±9% of BADJs. Notably, CCA staining appears to be less abundant in DPCs than in Clara cells. DPCs are not identified in alveolar spaces, bronchioles, large airways, or in the trachea. Rarely, DPCs are found in the proximal portion of the terminal bronchiole (data not shown). Staining for the neuroendocrine cell marker CGRP was not observed at the BADJ (data not shown). Because the data described below provide evidence that DPCs function as stem cells for Clara cells and alveolar cells, DPCs are referred to herein as bronchioalveolar stem cells (BASCs).

BASCs Are Expanded in Lung Tumorigenesis

Identification of BASCs at the BADJ placed them at a putative site of tumorigenesis; therefore, BASCs may be important in lung adenocarcinoma initiation. Serial section analysis indicated that Lox-K-ras adenomas often arose near terminal bronchioles, and lesions present at early time points after oncogenic K-ras activation appeared to be continuations of bronchiolar hyperplasia and alveolar hyperplasia arising in terminal bronchioles (Jackson et al. (2001) Genes Dev. 15, 3243-3248).

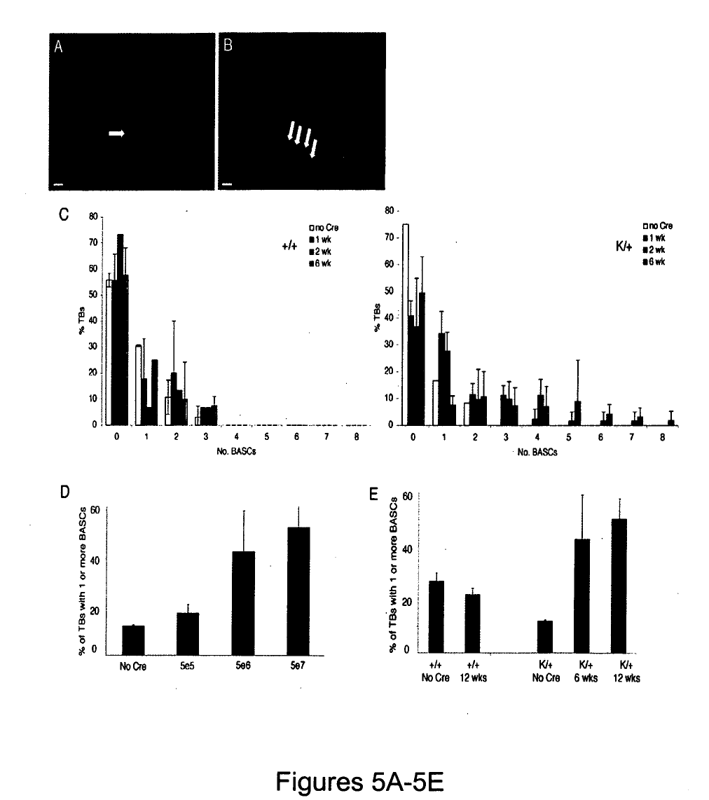

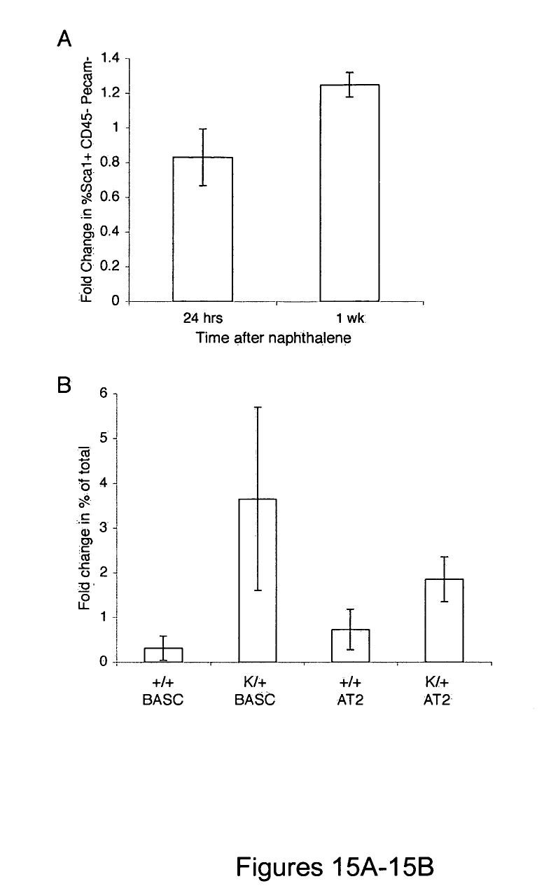

Supporting their role in tumorigenesis, immunofluorescence (IF) showed that BASC numbers were increased in the earliest tumorigenic lesions in Lox-K-ras mice. Whereas the majority of wild-type terminal bronchioles contained one to three BASCs with or without AdCre (FIGS. 5A and 5C), up to four BASCs at a single BADJ were detected 1 week after AdCre in 2% of Lox-K-ras terminal bronchioles (FIGS. 5B and 5C). The distribution of BASCs continued to increase 2 weeks after AdCre; seven BASCs were observed at single BADJs, and 16% of TBs had four to seven BASCs (p=1.7e⁻⁶ compared to wild-type controls) (FIG. 5C). Verifying IF results, a 3.6-fold increase in BASCs in Lox-K-ras mice was observed by FACS analysis 5 days after AdCre administration (p=0.009) (FIGS. 15A and 15B). These data show that BASC expansion was coincident with the formation of AAH, the precursor lesion for adenocarcinoma (Jackson et al. (2001), supra). Furthermore, delivery of increased titer of AdCre, which resulted in increased tumor number (Jackson et al. (2001) supra), was also correlated to an increase in BASC incidence (p=1.7e⁻¹²; FIG. 5D), linking BASC expansion to tumor number. BASC numbers further increased during tumor progression; 37% of Lox-K-ras terminal bronchioles had four to eight BASCs 6 weeks after Cre (p=5.4e⁻⁶; FIG. 5C) and BASCs were also expanded 12 weeks after Cre (p=1.3e⁻⁶; FIG. 5E).

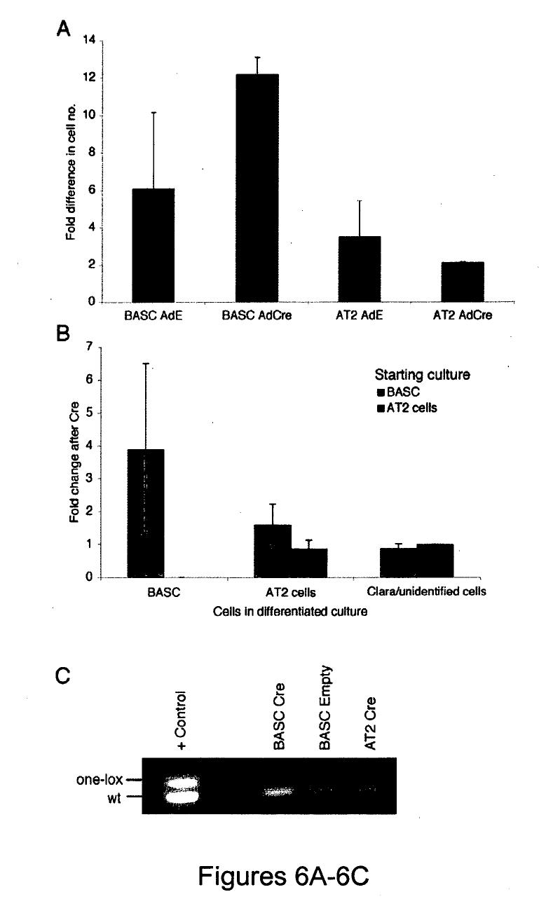

Consistent with BASC expansion being a key, early event in tumorigenesis, we observed a specific effect of K-ras G12D activation on the proliferation and differentiation of BASCs. LSL-K-ras G12D BASC cultures treated with AdCre underwent a 2.5-fold expansion in cell number compared to control cultures (p=0.05; FIG. 6A). In contrast, AdCre-infected AT2 cultures did not show a significant change in cell number. AdCre-infected BASC cultures exhibited reduced differentiation; the percentage of Matrigel-grown BASC cultures composed of Sca-1^{pos} CD45^{neg} Pecam^{neg} cells was 4-fold increased compared to AdEmpty-infected cultures, there was a 1.6-fold increase in AT2 cells, and a decrease in other cells was observed (FIG. 6B). These findings were similar to the changes in abundance of BASCs and AT2 cells observed by FACS after K-ras G12D activation in vivo (FIGS. 15A and 15B). Importantly, BASCs did not arise in AdCre-treated AT2 cell cultures. Our observations were not due to inherent differences in ability to infect BASC and AT2 cell cultures, as they exhibited similar efficiency for recombination of the K-ras allele (FIG. 6C). These data suggested that the first outcome of K-ras G12D activation on lung epithelia is an expansion of BASC number and subsequent differentiation toward the alveolar lineage.

BASC Stimulation in Vivo Affects Tumorigenesis

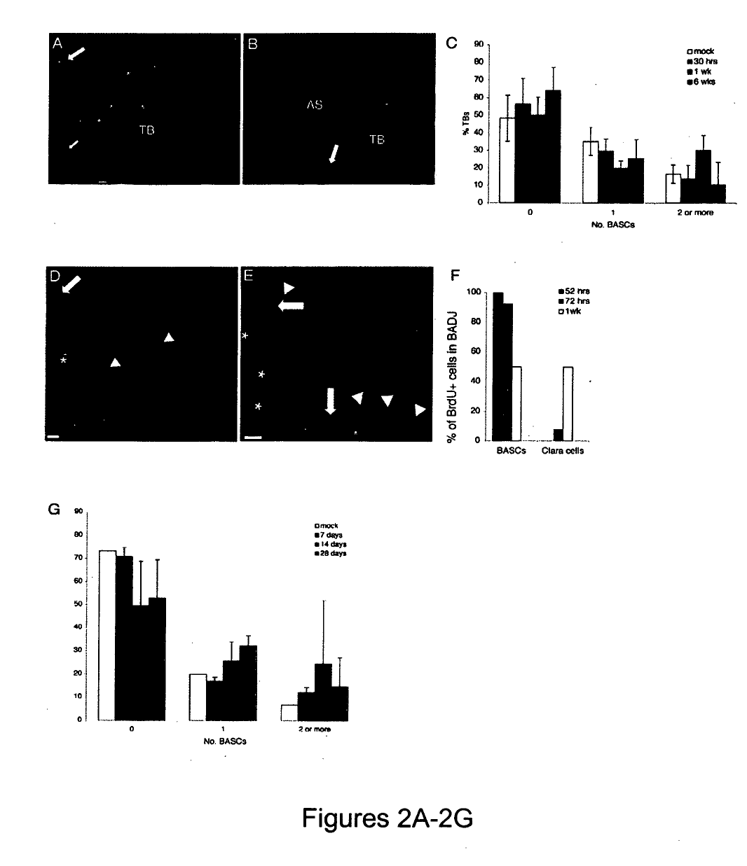

We reasoned that the cellular requirements for adenocarcinoma formation could be dissected further by combining K-ras G12D activation with naphthalene treatment, which stimulates proliferation of BASCs in vivo (see FIG. 2). LSL-K-ras G12D mice and wild-type controls were treated with naphthalene; tumorigenesis was initiated by administering AdCre 24 hr to 3 days later, and mice were euthanized after 6 weeks to determine tumor incidence and size.

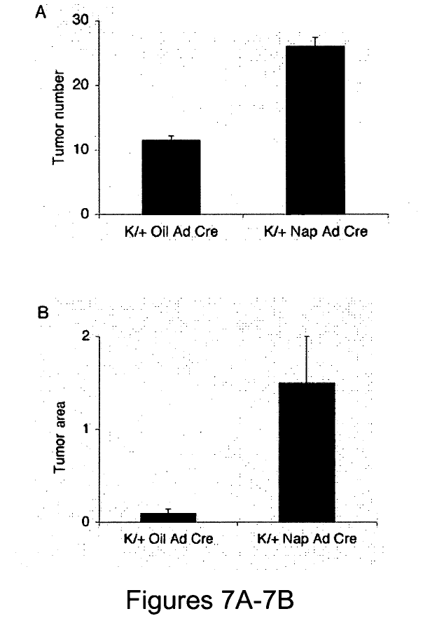

Naphthalene administration resulted in an increase in tumor number and area, further implicating BASCs in tumor initiation. In three independent experiments, naphthalene treatment was correlated with an increase in tumor number; 1.3-, 2.2-, and 2.3-fold more tumors were observed in Lox-K-ras mice treated with naphthalene before AdCre compared to controls (p=0.04; FIG. 7A). An even greater impact on tumor area was associated with naphthalene treatment; adenomas comprised a 6-, 12-, and 15-fold greater proportion of total lung area when initiated after airway damage in the three experiments (p=0.009; FIG. 7B). Importantly, increased tumorigenicity was not attributable to more effective AT2 cell infection after naphthalene treatment. Infection with AdGFP followed by IF for SPC or CCA 24 hr later indicated that there was no significant increase in the incidence of GFP^{pos} AT2 cells in control versus naphthalene-treated animals (7.8% versus 10.5% of infected cells were SP-C^{pos}).

### EXAMPLE 4

### Measuring BASC Number in Vivo

The present invention also features methods of measuring BASC number in vivo, by administering a compound to a subject that specifically binds BASCs, and then detecting the compound in the subject, where an increase or decrease amount of the compound detected in the lung of the subject relative to a control indicates a change in the number of BASCs. As an increased number of BASCs are associated with early stages of lung cancer, an increase in BASC number can be diagnostic of early stage lung cancer, or an increased propensity towards developing lung cancer. Early detection is critical for increasing survival rates of individuals with lung cancer. In vivo detection of BASC as described allows for such detection to be done non-invasively.

Compounds for Use in BASCs Detection Assays.

Detection of BASC cells may be by methods standard in the art, such as antibodies that specifically bind markers of BASCs (e.g., CD34 or Sca-1). Production methods for these antibodies are standard in the art and described herein.

Antibodies

To generate antibodies, an appropriate polypeptide may be expressed as a C-terminal fusion with glutathione S-transferase (GST) (Smith D. B. & Johnson, K. S., (1988) Gene 67, 31-40). The fusion protein is purified on glutathione-Sepharose beads, eluted with glutathione, cleaved with thrombin (at the engineered cleavage site), and purified to the degree necessary for immunization of rabbits. Alternatively, cells may be used for immunization, as is standard in the art. Primary immunizations are carried out with Freund's complete adjuvant and subsequent immunizations with Freund's incomplete adjuvant. Antibody titres are monitored by Western blot and immunoprecipitation analyses using the thrombin-cleaved polypeptide fragment of the GST fusion protein. Immune sera are affinity purified using CNBr-Sepharose-coupled polypeptide. Antiserum specificity is determined using a panel of unrelated GST proteins.

As an alternate or adjunct immunogen to GST fusion proteins, peptides corresponding to relatively unique immunogenic regions of a polypeptide describe herein may be generated and coupled to keyhole limpet hemocyanin (KLH) through an introduced C-terminal lysine. Antiserum to each of these peptides is similarly affinity purified on peptides conjugated to BSA, and specificity tested in ELISA and Western blots using peptide conjugates, and by Western blot and immunoprecipitation using the polypeptide expressed as a GST fusion protein.

Alternatively, monoclonal antibodies which specifically bind any one of the polypeptides or cells (e.g., BASCs) described herein are prepared according to standard hybridoma technology (see, e.g., Kohler et al. (1975) Nature 256, 495; Kohler et al., (1976) Eur J Immunol 6, 511; Kohler et al., (1976) Eur J Immunol 6, 292; Hammerling et al., In Monoclonal Antibodies and T Cell Hybridomas, Elsevier, N.Y., 1981; Ausubel et al. (Current Protocols in Molecular Biology, Wiley Interscience, New York, 1997)). Once produced, monoclonal antibodies are also tested for specific recognition by Western blot or immunoprecipitation analysis (by the methods described in Ausubel et al., supra). Antibodies which specifically recognize such a polypeptide are considered to be useful in the invention; such antibodies may be used, e.g., in an imaging technique. Alternatively monoclonal antibodies may be prepared using the polypeptides or BASCs described above and a phage display library (Vaughan et al., (1996) Nature Biotechnol 14, 309-314).

Preferably, antibodies of the invention are produced using fragments of the polypeptides described herein or cells which lie outside generally conserved regions and appear likely to be antigenic, by criteria such as high frequency of charged residues. In one specific example, such fragments are generated by standard techniques of PCR and cloned into the pGEX expression vector (Ausubel et al., supra). Fusion proteins are expressed in E. coli and purified using a glutathione agarose affinity matrix as described in Ausubel et al. (supra). To attempt to minimize the potential problems of low affinity or specificity of antisera, two or three such fusions are generated for each polypeptide, and each fusion is injected into at least two rabbits. Antisera are raised by injections in a series, preferably including at least three booster injections.

Antibodies against any of the cells (e.g., BASCs) or polypeptides described herein may be employed to diagnose, treat, or prevent lung diseases such as lung cancer. Antibodies may be further derivatized with markers detectable by imaging techniques (e.g., isotopic labels for scintigraphic techniques) described below.

Compounds that Specifically Bind BASCs

Any compound that specifically binds a BASC may be used in the detection methods of the invention. Such compouonds may also be derivatized with markers detectable by imaging techniques (e.g., isotopic labels for scintigraphic techniques) described below.

Imaging Techniques

Any standard imaging technique can be used in to measure changes in BASC population. Those skilled in the art will know which types of imaging device can be used with specific (e.g., radiographic) agents.

Scintigraphy

Scintigraphy involves the use of radioactive isotopes to diagnose and treat various diseases. It has applications in neurology, cardiology, oncology, endocrinology, lymphatics, urinary function, gastroenterology, pulmonology, and other areas. Generally, the radioactive isotope is chemically bonded to a specific compound, and then injected intravenously. Different radioactive isotopes are used for different scintigraphy methods and three common methods are SPECT, PET, and planar scan (Anderson and Welch, Chem. Rev. 99:2219-2234, 1999; Langer and Beck-Sickinger, Curr. Med. Chem. Anti-Canc. Agents 1:71-93, 2001).

SPECT (Single Photon Emission Computer Tomography) is a nuclear imaging technique that uses gamma rays. After injection of an isotope, a rotating gamma camera collects images 360 degrees around a patient, selecting only photons of certain energy. The images from various levels of the body are processed and recombined to form a 3D image.

PET (Positron Emission Tomography) is a nuclear imaging technique that uses radioactive isotopes that decay by positron emission, resulting in the release of two photons in opposite directions. A 360-degree scanner detects these photons, but only paired photons are processed. The safety of PET scans is higher than body CT, with radiation absorbed-from a PET scan at 5 mSv, whereas the radiation absorbed from a CT scan of the body ranges from 6-16 mSv.

Planar scanners were among the first generation of scintillation detecting devices and are currently used in many diagnostic procedures. They are in common use and yield two-dimensional images.

MRI

In addition to scintigraphy methods, magnetic resonance imaging (MRI) can also be used in the methods of the invention. MRI uses strong magnetic fields to generate three dimensional tomographic images of a patients bodies and does not require radioactivity.

### EXAMPLE 5

### Screening Methods to Identify Candidate Therapeutic Compounds

The present invention features methods of identifying candidate compounds that may be useful in diseases where BASC proliferation is increased (e.g., lung cancer) or decreased. Such methods involve contacting a BASC with a candidate compound, and measuring the rate of proliferation of the BASC, where an increase or decrease in BASC proliferation relative to a untreated BASC indicates that the compound is a candidate compound for the treatment of a disease associated with a change in BASC proliferation.

Screening Assays

Screening assays to identify compounds that increase or decrease BASC proliferation are carried out by standard methods. The screening methods may involve high-throughput techniques. In addition, these screening techniques may be carried out in cultured cells.

Any number of methods is available for carrying out such screening assays. According to one approach, candidate compounds are added at varying concentrations to the culture medium of BASC cells. Proliferation is then measured, for example, by an MTT assay (ATCC Bioproducts) to measure proliferation or by an apoptosis assay (e.g., TUNEL, Invitrogen Carlsbad, Calif.) to measure changes in cell death to determine if changes in cell proliferation have taken place. The level of proliferation in the presence of the candidate compound is compared to the level measured in a control culture medium lacking the candidate molecule. A compound which promotes an increase or a decrease BASC proliferation considered useful in the invention; such a molecule may be used, for example, as a therapeutic for a proliferative or other lung disorder (e.g., lung cancer and chronic lung diseases).

Alternatively, the assay can be carried out in animal (e.g., a mouse). In this case, a compound is administered to the animal by an appropriate means (e.g., intravenous, intratracheal, or oral administration). Proliferation of BASCs can be measured using histological techniques (e.g., those described herein). Again, a compound that, for example, decreases BASC proliferation, may be useful in the treatment of a disease such as lung cancer.

Optionally, compounds identified in any of the above-described assays may be confirmed as useful in delaying or ameliorating proliferation disorders in either standard tissue culture methods or animal models and, if successful, may be used as therapeutics for treating proliferative or other lung disorder (e.g., lung cancer and chronic lung diseases).

Small molecules provide useful candidate therapeutics. Preferably, such molecules have a molecular weight below 2,000 daltons, more preferably between 300 and 1,000 daltons, and most preferably between 400 and 700 daltons. It is preferred that these small molecules are organic molecules.

Test Compounds and Extracts

In general, compounds capable of treating a proliferative or other lung disorder (e.g., lung cancer and chronic lung diseases) are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and polynucleotide-based compounds. Synthetic compound libraries are commercially available. Alternatively, libraries of natural compounds in the form of bacterial, fungal, plant, and animal extracts are commercially available. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in treating proliferative or other lung disorder (e.g., lung cancer and chronic lung diseases) should be employed whenever possible.

When a crude extract is found to increase or decrease BASC proliferation, further fractionation of the positive lead extract is necessary to isolate chemical constituents responsible for the observed effect. Thus, the goal of the extraction, fractionation, and purification process is the characterization and identification of a chemical entity within the crude extract having activity that may be useful in treating a proliferative or other lung disorder (e.g., lung cancer and chronic lung diseases). Methods of fractionation and purification of such heterogenous extracts are known in the art. If desired, compounds shown to be useful agents for the treatment of a proliferative or other lung disorder (e.g., lung cancer and chronic lung diseases) are chemically modified according to methods known in the art.

### EXAMPLE 6

### Treatment of a Lung Disease

The present invention also features methods of treating a subject with lung diseases such as a proliferative disease or other lung disorder (e.g., lung cancer and chronic lung diseases) using a compound that increases or decreases proliferation of BASC cells. Certain embodiments include methods which decrease BASC proliferation and thus can be useful in treatment of, for example, lung cancer. Treatment can be administered by any suitable means including oral, parenteral (e.g., intravenously or intramuscularly), rectal, cutaneous, nasal, vaginal, inhalant, skin (patch), ocular, and intratracheal. The compounds used in the treatment of lung diseases can, for example, be compounds identified using the screening methods described herein.

Formulation of Pharmaceutical Compositions

The administration of any compound, such as those identified using the methods of the invention may be by any suitable means that results in a concentration of the compound that treats a lung disease (e.g., lung cancer or chronic lung disease). The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously or intramuscularly), rectal, cutaneous, nasal, vaginal, inhalant, skin (patch), ocular, or intratracheal administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions may be formulated to release the active compound immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agents of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the compound to a particular target cell type. Administration of the compound in the form of a controlled release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound is formulated with appropriate excipients into a pharmaceutical composition that, upon administration, releases the compound in a controlled manner. Examples include single or multiple unit tablet or capsule compositions, oil solutions, suspensions, emulsions, microcapsules, molecular complexes, microspheres, nanoparticles, patches, and liposomes.

Parenteral Compositions

The composition containing compounds described herein or identified using the methods of the invention may be administered parenterally by injection, infusion, or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active agent(s), the composition may include suitable parenterally acceptable carriers and/or excipients. The active agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions according to the invention may be in a form suitable for sterile injection. To prepare such a composition, the suitable active agent(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, dextrose solution, and isotonic sodium chloride solution. The aqueous formulation may also contain one or more preservatives (e.g., methyl, ethyl, or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Controlled Release Parenteral Compositions

Controlled release parenteral compositions may be in the form of aqueous suspensions, microspheres, microcapsules, magnetic microspheres, oil solutions, oil suspensions, or emulsions. The composition may also be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamnine), poly(lactic acid), polyglycolic acid, and mixtures thereof. Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters)) or combinations thereof.

Solid Dosage Forms for Oral Use

Formulations for oral use include tablets containing the active ingredient(s) in a mixture with non-toxic pharmaceutically acceptable excipients, and such formulations are known to the skilled artisan (e.g., U.S. Pat. No.: 5,817,307, 5,824,300, 5,830,456, 5,846,526, 5,882,640, 5,910,304, 6,036,949, 6,036,949, 6,372,218, hereby incorporated by reference). These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the compound in a predetermined pattern (e.g., in order to achieve a controlled release formulation) or it may be adapted not to release the agent(s) until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols, and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate, may be employed.

The solid tablet compositions may include a coating adapted to protect the composition from unwanted chemical changes, (e.g., chemical degradation prior to the release of the active substances). The coating may be applied on the solid dosage form in a similar manner as that described in Encyclopedia of Pharmaceutical Technology, supra.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate, or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus, or spray drying equipment.

Controlled Release Oral Dosage Forms

Controlled release compositions for oral use may, e.g., be constructed to release the compound by controlling the dissolution and/or the diffusion of the compound.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, DL-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax, and stearyl alcohol, carbopol 934, silicone, glyceryl tristearate, methyl acrylate-methyl methacrylate, polyvinyl chloride, polyethylene, and/or halogenated fluorocarbon.

A controlled release composition containing compounds described herein or identified using methods of the invention may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the composition with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Dosages

The dosage of any compound described herein or identified using the methods described herein depends on several factors, including: the administration method, the lung disease to be treated, the severity of the lung disease, whether the lung disease is to be treated or prevented, and the age, weight, and health of the subject to be treated.

With respect to the treatment methods of the invention, it is not intended that the administration of a compound to a subject be limited to a particular mode of administration, dosage, or frequency of dosing; the present invention contemplates all modes of administration, including intramuscular, intravenous, intraperitoneal, intravesicular, intraarticular, intralesional, subcutaneous, intratracheal, or any other route sufficient to provide a dose adequate to treat a lung disease. The compound may be administered to the subject in a single dose or in multiple doses. For example, a compound described herein or identified using screening methods of the invention may be administered once a week for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compound. For example, the dosage of a compound can be increased if the lower dose does not provide sufficient activity in the treatment of a lung disease (e.g., lung cancer or chronic lung disease). Conversely, the dosage of the compound can be decreased if the lung disease is reduced or eliminated.

While the attending physician ultimately will decide the appropriate amount and dosage regimen, a therapeutically effective amount of a compound described herein or identified using the screening methods of the invention, may be, for example, in the range of 0.0035 μg to 20 μg/kg body weight/day or 0.010 μg to 140 μg/kg body weight/week. Desirably a therapeutically effective amount is in the range of 0.025 μg to 10 μg/kg, for example, at least 0.025, 0.035, 0.05, 0.075, 0.1, 0.25, 0.5, 1.0, 1.5, 2.0, 2.5, 3.0, 3.5, 4.0, 5.0, 6.0, 7.0, 8.0, or 9.0 μg/kg body weight administered daily, every other day, or twice a week. In addition, a therapeutically effective amount may be in the range of 0.05 μg to 20 μg/kg, for example, at least 0.05, 0.7, 0.15, 0.2, 1.0, 2.0, 3.0, 4.0, 5.0, 6.0, 7.0, 8.0, 10.0, 12.0, 14.0, 16.0, or 18.0 μg/kg body weight administered weekly, every other week, or once a month. Furthermore, a therapeutically effective amount of a compound may be, for example, in the range of 100 μg/m² to 100,000 μg/m² administered every other day, once weekly, or every other week. In a desirable embodiment, the therapeutically effective amount is in the range of 1000 μg/m² to 20,000 μg/m², for example, at least 1000, 1500, 4000, or 14,000 μg/m² of the compound administered daily, every other day, twice weekly, weekly, or every other week.

### EXAMPLE 7

### Treatment of Lung Disease with BASCs

The present invention also features methods of treating lung disease, for example, chronic lung disease (e.g., obstructive pulmonary disease, cystic fibrosis, or emphysema) by administration of BASCs (e.g., human BASCs) to a subject in need of treatment. Such cells can be used to regenerate damage lung tissue. Administration of these cells may be using techniques standard in the art, such as intratracheal injection or pulmonary administration.

BASCs Respond to Bronchiolar and Alveolar Injury

Identification of BASCs at the BADJ localizes them in a putative stem cell niche. Previous studies showed that naphthalene-resistant Clara cells are present at the BADJ. Furthermore, the BrdU^{pos} CCA^{pos} cells identified during early airway renewal are located precisely where BASCs are found (Giangreco et al. (2002) Am. J. Pathol. 161, 173-182; Hong et al. (2001) Am. J. Respir. Cell Mol. Biol. 24, 671-681;Reynolds et al. (2000) Am. J. Pathol. 156, 269-278; Reynolds et al. (2000) Am. J. Physiol. Lung Cell Mol. Physiol. 278, 1-1256-1-1263).

To determine if airway damage affects BASCs, dual IF for CCA and SP-C is performed at various time points after naphthalene treatment. Although significant Clara cell loss is observed by 52 hr after naphthalene treatment, the number of BASCs did not significantly decrease at any time point (FIGS. 2A-2C and data not shown). The naphthalene-resistant population includes BASCs and Clara cells. However, in some terminal bronchioles, the only cells remaining are BASCs (data not shown). IF revealed that there is a significant increase in terminal bronchioles with two or more BASCs one week after naphthalene (16%±5% in controls to 30%±9% at 1 week, p=0.03; FIG. 2C). Consistent with IF, FACS analysis (described below; FIG. 3) shows that the abundance of BASCs did not change 24 hr after naphthalene but is 1.3-fold increased by 1 week after treatment (p=0.04). Six to seven weeks after naphthalene treatment, bronchiolar epithelium is restored (Giangreco et al. (2002) Am. J. Pathol. 161, 173-182; data not shown), and the number of BASCs per BADJ is then comparable to that in untreated lung (FIG. 2C). BrdU administered prior to euthanasia is used to determine if BASCs proliferate in response to naphthalene. In the BADJ, BrdU^{pos} cells are first detected at 52 hr after naphthalene, and all are SP-C^{pos} CCA^{pos} (FIGS. 2D and 2F). Seventy-two hours after naphthalene, CCA^{pos} SP-C^{neg} BrdU^{pos} cells are present adjacent to BrdU^{neg} BASCs (FIGS. 2E and 2F), suggesting that they arise from BASCs. BrdU^{pos} cells in the nonterminal bronchiolar epithelium at all time points are CCA^{pos} SP-C^{neg}, consistent with the report of distinct stem cell niches in proximal and terminal bronchioles (Giangreco et al. (2002) Am. J. Pathol. 161, 173-182). BASCs are BrdU^{neg} in normal, untreated lung (data not shown), whereas they proliferate during airway repair and are BrdU^{neg}, following its completion.

As these data point to a role for BASCs in terminal bronchiolar cell maintenance, the role of BASCs in response to alveolar damage is examined next. Bleomycin is administered intranasally, and at various time points after treatment, the incidence of BASCs is determined. Similar to their response to naphthalene, BASC numbers increase following bleomycin treatment. Significant increases are observed 14 days after bleomycin (p=0.01), when AT1 cell depletion became evident in previous studies (Aso et al. (1976) Lab. Invest. 35, 558-568). By contrast, BASC distribution is significantly different neither at 7 days after treatment, before substantial alveolar damage was documented, nor after 28 days, when effective repair is complete or long term damage is apparent (Aso et al. (1976) Lab. Invest. 35, 558-568; FIG. 2G).

Thus, BASCs play a role in both bronchiolar and alveolar cell injury repair and homeostasis.

### EXAMPLE 8

### Additional Experimental Procedures

Mice and Tissues

Lox-stop-lox K-ras G12D mice (Jackson et al. (2001) Genes Dev. 15, 3243-3248) and wild-type littermates were maintained in viral-free conditions on an F1 129 SvJ/C57BI6 background. Four- to eight-week-old mice were used for FACS or intranasal infections as described (Jackson et al. (2001) Genes Dev. 15, 3243-3248). Naphthalene (Aldrich) was dissolved in MAZOLA corn oil and injected i.p. at 275 mg/kg using sex- and background-matched control mice. Bleomycin (Sigma; 40 μl of 1.88 U/ml in PBS) was administered intranasally. 30 mg/kg BrdU in PBS was injected i.p. 2 hr before euthanasia for proliferation studies. Tissue preparation was as described (Jackson et al., 2001, supra). BASCs were quantified by scoring sections from at least three mice for each condition. Bioquant Software was used to quantify tumor area.

Immunofluorescence

Immunostaining of tissues and cells was performed as previously described (Jackson et al., 2001) using antisera raised against mouse CCA (gift); proSP-C (RDI); proSP-C clone 7705, CC10, CD34 clone C-18, and aquaporin 5 (Santa Cruz); proSP-C (Chemicon clone 3786); CGRP and acetylated tubulin (Sigma); CD34 Mecl4.7 (Abcam); Sca-1 (R&D Systems); BrdU (Becton Dickinson); and donkey Alexafluore secondary antibodies (Molecular Probes). Sorted or cultured cells were stained after cytospin at 600 rpm for 3 min and fixation and permeabilization with CytoFix/CytoPerm (Pharmingen). Triple-color microscopy and imaging were performed with a Nikon Eclipse E600 and a Spot cooled CCD camera and software. Four-color microscopy and deconvolution were performed with a Zeiss Axioplan II, cooled CCD camera, and OpenLab software. Images were processed with Adobe Photoshop.

Epithelial Cultures

Cells were plated in DME/HEPES/10% FBS on 96-well plates coated with 100 pi Matrigel (Becton Dickinson) or irradiated DR4 MEFs. Matrigel cultures were fixed on plates or treated with collagenase/dispase for cytospin, IF, and FACS. For limiting dilution analysis, 1, 10, 100, or 1000 cells were sorted onto feeders to assess colony formation 1 week later. Regression analysis was performed to calculate the x value at which they value is 37%, which correlates with each colony arising from a single cell (Tropepe et al. (2000) Science 287, 2032-2036). For serial passage, wells containing a single colony were trypsinized, and single cell suspensions were plated onto fresh feeder-coated wells for the next passage. Colony number was assessed after 1 week, and the process was repeated. Matrigel cultures were infected with 1 e6 PFU AdCre or AdEmpty after 1-3 days in culture and analyzed by FACS 7 days later.

Statistics

Statistical analyses were performed using Mstat software (Norman Drinkwater, McArdle Laboratory for Cancer Research, University of Wisconsin). For comparison of the distribution of BASC number per terminal bronchiole, the Cochran-Armitage test was performed, and data with a trend p value of 0.05 or less were considered significant. Other analyses were done using the Wilcoxon rank sum test or Fisher's exact test as appropriate.

All patents, patent applications, and publications mentioned in this specification are herein incorporated by reference to the same extent as if each independent patent, patent application, or publication was specifically and individually indicated to be incorporated by reference.

What is claimed is:

1. A method of isolating BASCs, said method comprising:
(a) isolating cells from a lung of an organism;
(b) selecting from said cells a population which lacks markers for both hematopoietic and endothelial cells; and
(c) selecting, from said population from (b), a second population of cells containing a stem cell marker, thereby isolating BASCs.

2. The method of claim 1, wherein said organism is a mouse or a human.

3. The method of claim 1, wherein said step (b) or said step (c) selecting is performed using FACS.

4. The method of claim 1, wherein said method further comprises removal of red blood cells by lysis.

5. The method of claim 1, wherein said stem cell marker is Sca-1 or CD34.

6. The method of claim 1, wherein said hematopoietic cell marker is CD45 or Ter119.

7. The method of claim 1, wherein said endothelial cell marker is Pecam.

8. The method of claim 1, further comprising (d) selecting from said second population a third population using an additional marker.

9. The method of claim 8, wherein said additional marker is CD34 or Sca-1.

10. The method of claim 1, further comprising (d) growing the cells selected in step (c) in a feeder cell culture.

11. A multipotent stem cell isolated from lung, wherein said stem cell expresses at least one of Sca-1, SP-C, CCA, and CD34.

12. The stem cell of claim 11, wherein said stem cell expresses at least two of Sca-1, SP-C, CCA, and CD34.

13. The stem cell of claim 11, wherein said stem cell expresses at least three of Sca-1, SP-C, CCA, and CD34.

14. The stem cell of claim 11, wherein said stem cell expresses Sca-1, SP-C, CCA, and CD34.

15. The stem cell of claim 11, comprising a genetic modification.

16. A method of identifying an increase or decrease in the number of BASCs in a lung of a subject, said method comprising:
(a) administering to said subject a compound that specifically binds a BASC; and
(b) detecting said compound in said lung of said subject, wherein an increase or decrease in the amount of said compound detected in the lungs of said subject as compared to an amount in a control subject indicates an increase or decrease in the number of BASCs in the lung of said subject.

17. The method of claim 16, wherein said compound is an antibody, or a BASC-binding fragment thereof.

18. The method of claim 17, wherein said antibody is antibody is an anti-CD34 antibody, or a CD34-binding fragment thereof.

19. The method of claim 16, wherein said administering is intravenous or intratracheal.

20. The method of claim 16, wherein said detecting is performed by an imaging technique.

21. The method of claim 20, wherein said imaging technique is selected from the group consisting of planar scan, PET, SPECT, MRI, and CT.

22. The method of claim 16, wherein said subject is a human.

23. A method of identifying a candidate compound for treating a subject with a disease associated with a change in BASC proliferation or differentiation, said method comprising:
(a) contacting a BASC with a compound; and
(b) measuring proliferation and/or differentiation of said BASC, wherein an increased or decreased level of BASC proliferation or differentiation in the presence of said compound relative to the level in the absence of said compound identifies said compound as a candidate compound for treating a subject with a change in BASC proliferation.

24. The method of claim 23, wherein said change in BASC proliferation is an increase in BASC proliferation.

25. The method of claim 24, wherein said increase is associated with lung cancer.

26. The method of claim 23, wherein said BASC is a mammalian cell.

27. The method of claim 26, wherein said BASC is a human cell.

28. The method of claim 23, wherein said BASC is in vitro.

29. The method of claim 23, wherein said BASC is in vivo.

30. The method of claim 29, wherein said BASC is in a mouse.

31. A method of treating a subject having a lung disease, said method comprising administering BASCs to said subject in an amount sufficient to treat said lung disease.

32. The method of claim 31, wherein said administration is intratracheal.

33. The method of claim 31, wherein said subject is a human.

34. The method of claim 31, wherein said BASC is a human BASC.

35. The method of claim 31, wherein said lung disease is chronic, obstructive pulmonary disease, cystic fibrosis, or emphysema.
